(19) United States
(12) Patent Application Publication
(10) Pub. No.: US 2022/0394827 A1
(43) Pub. Date: Dec. 8, 2022

(54) NON-CONTACT RADIO-FREQUENCY HEATING

(71) Applicants: Airity Technologies, Inc., Redwood City, CA (US); Teleflex Life Sciences Limited, Valletta (MT)

(72) Inventors: Luke Christopher Raymond, Redwood City, CA (US); Wei Liang, Redwood City, CA (US); Vladimir Gusiatnikov, Redwood City, CA (US); Johan Oscar Lennart Andreasson, Redwood City, CA (US); Bang Quoc Vu, Redwood City, CA (US)

(73) Assignees: Airity Technologies, Inc., Redwood City, CA (US); Teleflex Life Sciences Limited, Valletta (MT)

(21) Appl. No.: 17/774,559

(22) PCT Filed: Oct. 26, 2020
(86) PCT No.: PCT/US2020/057339

§ 371 Date: May 5, 2022

Related U.S. Application Data
(60) Provisional application No. 62/933,173, filed on Nov. 8, 2019.

(51) Int. Cl. H05B 6/50; H05B 1/02

(52) U.S. Cl. CPC H05B 6/50; H05B 1/025; H05B 2203/021

(57) ABSTRACT

A control unit and methods for operating the control unit to perform non-contact radio-frequency (RF) heating of a fluid flowing through or contained within a non-contact radio-frequency heating element.

### BACKGROUND

Various types of fluids, such as saline, are routinely introduced in some format into a patient, such as a human or an animal, as part of a medical procedure or for various medical reasons. Because these fluids are being introduced for example into the patient's bloodstream, the fluid needs to be provided in a sterile environment, and must not be contaminated as part of the process of transferring the fluid from the fluid's original packaging or container to the patient. In addition, to decrease any level of discomfort or chilling of the patient as a result of the introduction of the fluid into the patient's bloodstream, the fluid is often warmed, for example using a heating device, to a temperature above a normal room temperature and to a temperature that more closely corresponds to the body temperature of the patient. The heating of the fluid must be performed in a manner that does not cause or contribute to any contamination of the fluid prior to or following the introduction of the fluid into the patient.

### BRIEF DESCRIPTION OF THE DRAWINGS

Aspects/Embodiments of the disclosure may be better understood by referencing the accompanying drawings.

FIG. 1 illustrates a schematic block diagram of an illustrative non-contact radio-frequency heating system according to at least one embodiment.

FIG. 2 illustrates a schematic block diagram of a non-contact radio-frequency heating system according to at least one embodiment.

FIGS. 3A-3C illustrate graphical depictions of various electrical output waveforms that may be generated and applied to one or more electrodes of a non-contact radio-frequency heating element according to at least one embodiment.

FIG. 4 illustrates a schematic block diagram of a non-contact radio-frequency heating system according to at least one embodiment.

FIG. 4A illustrates a schematic block diagram including a non-contact radio-frequency heating element according to at least one embodiment.

FIGS. 5A-5C illustrate side, top, and bottom views, respectively, of an electrical waveform generator/power-delivery circuitry according to at least one embodiment.

FIG. 6 illustrates an electrical schematic diagram of an embodiment of electrical circuitry for a non-contact radio-frequency heating control unit according to at least one embodiment.

FIG. 7 illustrates a flowchart for a method for non-contact radio-frequency heating control according to at least one embodiment.

The drawings are provided for the purpose of illustrating example embodiments. The scope of the claims and of the disclosure are not necessarily limited to the systems, apparatus, methods, or techniques, or any arrangements thereof, as illustrated in these figures. In the drawings and description that follow, like parts are typically marked throughout the specification and drawings with the same or coordinated reference numerals. The drawing figures are not necessarily to scale. Certain features of the invention may be shown exaggerated in scale or in somewhat schematic form, and some details of conventional elements may not be shown in the interest of clarity and conciseness.

### DETAILED DESCRIPTION

The description that follows includes example systems, methods, techniques, and program flows that illustrate and describe various embodiments of a non-contact radio-frequency (RF) heating element and a control unit for operating a non-contact RF heating element. However, it is understood that this disclosure may be practiced without these specific details. For instance, this disclosure refers to non-contact RF heating applied to a flow-through non-contact RF heating element for use in medical procedures involving the heating of fluid for introduction into a patient in illustrative examples. However, aspects of this disclosure may be applied to other examples of systems used to heat a fluid, both in a flow-through and in static reservoir arrangements. In certain examples illustrated and described throughout the disclosure, well-known instruction instances, protocols, structures and techniques have not necessarily been shown in detail in order not to obfuscate the description.

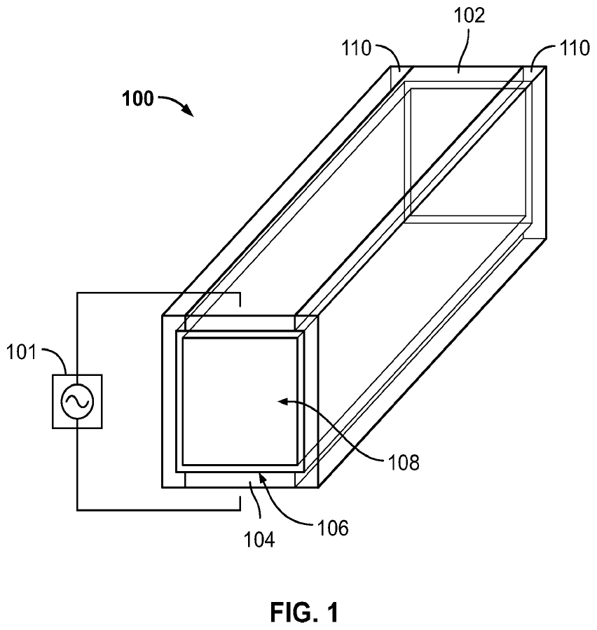

FIG. 1 illustrates a schematic block diagram of an illustrative non-contact radio-frequency heating system 100 (hereinafter “system 100”), according to at least one embodiment. As shown in FIG. 1, system 100 includes an electrical power source 101 having electrical connections to a first electrode 102 and to a second electrode 104. Electrodes 102 and 104 may be formed from an electrically conductive material, such as copper, or another electrically conductive metal, and may be spaced apart from one another by an area that includes a hollow cavity forming and at least partially enclosing a fluid passageway 108. Fluid passageway 108 may be configured to receive a flow of fluid, such as saline, or another fluid intended to be introduced into a patient, such as a human or an animal, after passing through the fluid passageway and being heated to a desired temperature during the time the fluid is transported through the passageway. The heating of the fluid flow is accomplished by the application of electrical energy provided to electrodes 102, 104 from electrical power source 101. Electrodes 102 and 104 may be referred to as one example of a set of electrodes. Electrical energy provided to electrodes 102, 104 is operable to produce an electromagnetic field in the area between the electrodes, which includes fluid passageway 108, and to generate non-contact RF heating of a fluid that is flowing through or that is contained within the fluid passageway, without having any direct physical contact with or being immersed into the fluid that is flowing through the fluid passageway.

In system 100, a dielectric barrier 106 at least partially surrounds the fluid passageway 108, and isolates the fluid passageway from electrodes 102, 104 so that the fluid passing through fluid passageway 108 is not brought into contact with the electrodes. Dielectric barrier 106 may be formed from an insulating material, such as but not limited to a plastic material such as polyimide. In various embodiments, dielectric barrier 106 may be part of the heating element body, and may be configured as a disposable sterile insert, such as a catheter, that is inserted within and extends through fluid passageway 108 to provide a sterile environment for the fluid to flow through while flowing through fluid passageway. In some embodiments, dielectric barrier 106 is part of the sterile environment used in contact with and to provide a conduit for the flow of fluid through the fluid passageway 108, and is removable and disposable after use in a fluid warming procedure utilizing system 100. A heating element body, such as body 110, may be configured to hold the electrodes 102, 104 in a position spaced apart from one another and proximate to fluid passageway 108. Electrode 102 and/or electrode 104 may be partially or wholly embedded within body 110 in some embodiments in order to maintain the proper positioning of the electrodes relative to each other and to dielectric barrier 106 and fluid passageway 108.

As further described below, system 100 may be configured to provide and control electrical energy that is output from the electrical power source 101 and provided to electrodes 102, 104, in order to provide a controlled heating of the fluid flowing through passageway 108, such as a fluid intended for introduction into a patient, while the fluid flows through or is present within the fluid passageway. System 100 may be further configured to warm the flow of fluid through fluid passageway 108 while maintaining a sterile environment with respect to any of the passageways and fluid conduits that come into direct contact with the fluid being heated subsequent to the introduction into the patient.

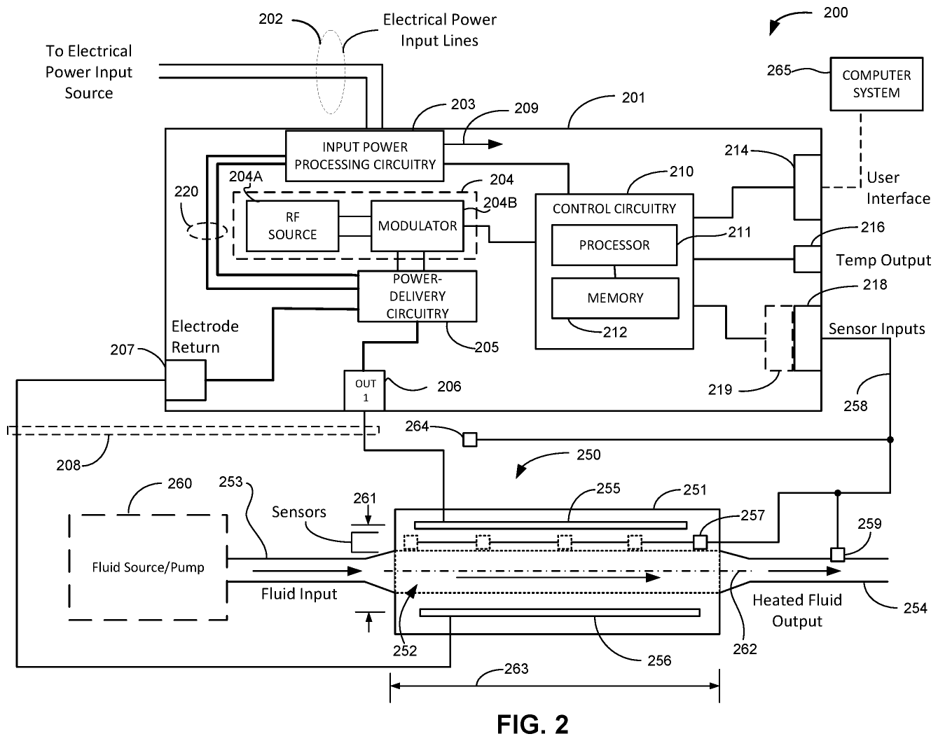

FIG. 2 illustrates a schematic block diagram of a fluid non-contact radio-frequency heating system 200 (hereinafter “system 200”) according to at least one embodiment. As shown in FIG. 2, system 200 includes a non-contact RF heating element 250 (hereinafter “element 250”) electrically coupled to a non-contact RF heating control unit 201 (hereinafter “control unit 201”). System 200 is configured to provide a controlled level of non-contact RF heating to a flow of fluid passing through element 250 by non-contact RF heating of the fluid flow using electrical energy provided and controlled by control unit 201 and applied to one or more sets of electrodes included in element 250, as further described below.

Element 250 includes a heating element body 251 (hereinafter “body 251”) having a first end coupled to a fluid input conduit 253 and a second end that is opposite the first end, the second end coupled to a fluid output conduit 254. A hollow passageway 252 extends from the first end to the second end of the body 251, forming a fluid passageway to transport a flow of fluid entering the first end of body 251 as provided by the fluid input conduit 253 to the second end of the body and to the outlet provided by fluid output conduit 254. Element 250 further includes one or more sets of electrodes positioned within body 251, the electrodes positioned proximate to passageway 252, and sealed from passageway 252, for example by a portion of the body 251, so that the electrodes will not come into contact with the fluid flowing through the passageway. Embodiments of passageway 252 are not limited to being formed as a single straight passageway, and in various embodiments may include a set of parallel passageways, or a single passageway that winds along, for example in a serpentine path or other non-linear path, through the body 251 of element 250.

As illustrated in FIG. 2, element 250 includes a first electrode 255 embedded within body 251 and positioned above passageway 252, and a second electrode, return electrode 256, also embedded within body 251 and positioned below passageway 252 and on the opposite side of the passageway with respect to the position of first electrode 255. Electrode 255 and return electrode 256 have respective surfaces facing the passageway 252 that that are spaced apart for one another by a distance 261. Distance 261 is not limited to a particular distance or range of distances, and in various embodiments includes a distance value in a range of 1 to 10 millimeters, inclusive. Electrode 255 and return electrode 256 in various embodiments are flat planar structures that extend parallel to each other and extend along some length of a longitudinal axis 262 of element 250. However, the configurations of electrode 255 and return electrode 256 are not limited to being shaped as flat planar structures, and may be formed into other shapes, such as but not limited to curved arch-shaped structures that extend radially around at least some portion of longitudinal axis 262 at some radial distance away from the longitudinal axis and extending along at least some portion of the longitudinal axis while remaining physically separated and electrically isolated from one another. Other arrangements for electrode 255 and return electrode 256 are possible and are contemplated for use in system 200. Further, as illustrated in FIG. 2 element 250 has a horizontal orientation along longitudinal axis 262. However, the orientation of a longitudinal axis, and thus the orientation of passageway 252 and/or a plurality of passageways included in an element such as element 250, is not limited to any particular orientation. The orientation of element 250 is not limited to a horizontal orientation while the element is coupled to a control unit and is being used in a RF heating application. In various embodiment, the orientation the RF heating element may include any orientation, including horizontal orientations, vertical orientations, or any angular orientation between a horizontal and vertical orientation.

Electrical energy provided by control unit 201 to electrode 255 and return electrode 256 may establish an electromagnetic field in an area between the electrodes, and thus be imposed onto a fluid included within passageway 252. The field established between the electrodes may then induce non-contact RF heating of the fluid included in the passageway. By controlling the amount and format to the electrical energy provided to electrode 255 and return electrode 256, control unit 201 may be configured to controllably heat a flow of fluid passing through passageway 252 of element 250. In various embodiments, the fluid to be heated is saline, or a saline solution, which is being provided as a non-limiting example of a fluid that may be introduced into a patient after passing through element 250 and being heated to a desired temperature before being introduced into the patient. Heating of the saline may reduce patient discomfort related to the introduction of the heated fluid introduced into the patient at a body temperature or a near body temperature as opposed to a lower temperature, such as a room temperature where the saline fluid is initially stored and introduced into system 200. In addition, because the saline solution is being provided to the patient and in a medical setting, it is important that the heating of the saline be accomplished without contamination of the saline as part of the heating process. As show in FIG. 2, because electrode 255 and return electrode 256 are not in contact with the flow of fluid through passageway 252, but instead are configured to provide non-contact RF heating to heat the flow of saline through element 250, system 200 provides a system and method for heating the fluid while maintaining a sterile environment with respect to any of the fluid passageway(s) that might come into contact with the fluid.

In various embodiments, element 250 of system 200 is configured to couple to a fluid source 260, wherein fluid source 260 may include a pump or other mechanism to produce a flow of fluid, such as a flow of saline, to fluid input conduit 253. Fluid input conduit 253 is coupled to the first end of body 251, and is in fluid communication with passageway 252. A flow of fluid, such as saline provided by fluid source 260, may flow through passageway 252 and between electrode 255 and return electrode 256, and exit body 251 through fluid output conduit 254. As the fluid flows through passageway 252, electrical energy under the control of control unit 201 may be provided to electrode 255 and return electrode 256, and produce non-contact RF heating of the fluid within passageway 252. One or more sensors, such as temperature sensor 257, may be positioned proximate to passageway 252, and may be configured to sense the temperature of the flow of fluid as the fluid passes through and exits passageway 252. The sensor(s) generate one or more sensor output signals that are indicative of the sensed temperature of the fluid passing through and/or exiting passageway 252, and provide the output signal(s) to a sensor input 218 of control unit 201, for example though sensor input lines 258. In some embodiments, sensor input 218 may include or be coupled to a multiplexer 219 configured to multiplex a plurality of input signals from multiple sensors into control circuitry 210, for example using some predefined sampling rate. Control unit 201 may be configured to receive and process the sensor input signal(s) related to temperature of the fluid, and to further control the output of electrical energy being provided to electrode 255 and return electrode 256 by controlling the electrical output being provided to electrode output terminal 206 and electrode return terminal 207 of the control unit.

In addition to temperature sensing, one or more other types of sensors, such as one or more flow sensors illustratively represented by sensor 259, and one or more ambient temperature sensors illustratively represented by sensor 264, may be included in system 200 to provide additional feedback to control unit 201. In various embodiments, flow sensor 259 is configured to determine a flow rate or a flow volume passing by the sensor, and provide an output signal to control unit 201 indicative of the flow rate or the volume of flow passing by the sensor. This flow rate/flow volume information may be received by control unit 201, and further incorporated into the control of the electrical energy being provide by the control unit to element 250 in order to maintain the temperature control of the flow of fluid passing through element 250 in a desired manner.

In various embodiments, ambient temperature sensor 264 is configured to determine an ambient temperature in one or more areas outside element 250, such as an ambient temperature of the area where the fluid source 260 is located, and/or an ambient temperature in the area where the fluid output conduit 254 passes between the element 250 and the point where the fluid is introduced into a patient. Ambient temperature sensor 264 may be configured to generate and to provide an output signal to control unit 201 indicative of the ambient temperature in one or more areas located outside of element 250. This ambient temperature information may be received by control unit 201, and further incorporated into the control of the electrical energy being provide by the control unit to element 250 to maintain the temperature control of the flow fluid passing through element 250 in a desired manner.

As shown in FIG. 2, control unit 201 includes an input power processing circuitry 203, an electrical waveform generator 204 including a radio-frequency(RF) source 204A and a modulator 204B, a power-delivery circuitry 205, and control circuitry 210. Embodiments of control unit 201 may include less or more components, and may include components arranged and coupled in a manner that is different from or varies in some degree or manner from the embodiment shown for system 200 and control unit 201. Variations of the number, types, and arrangements of these components are contemplated by the embodiments of non-contact RF heating control units as described throughout this disclosure, and any equivalents thereof.

As illustrated for system 200, input power processing circuitry 203 is coupled to at least one electrical power input source (not specifically shown in FIG. 2) through electrical power input lines 202. The electrical power input that may be provided to control unit 201 is not limited to any particular type or configuration of electrical power input. In various embodiments, the electrical power input may be a standard electrical power configuration that is provided by a private or government agency in a region where system 200 is being operated. For example, the electrical power input source may be a standardized alternating current (AC) 120 volt/60 hertz line voltage typical of electrical power provided in the United States. In other embodiments, the electrical power input may be a direct current (DC) input supply, for example from a battery or from an electrical power supply. In various embodiments, multiple power sources may be coupled to electrical power input lines 202. For example, lines 202 may be coupled to a conventional AC power source as the main power source, but also coupled to a backup power supply, such as a battery operated supply or a generator, which is configured to provide electrical power to lines 202 in the event of an electrical power failure of the main power source.

Regardless of the power input configuration, input power processing circuitry 203 may be configured to perform conditioning of the incoming electrical power to provide electrical power that is coupled to the electrical components and devices included in control unit 201, including the electrical waveform generator 204, control circuitry 210, and power-delivery circuitry 205. For the sake of clarity and simplicity, actual lines showing the specific power connections between the electrical components and devices of control unit 201 and the input power processing circuitry 203 may not be illustrated in FIG. 2, but are illustratively represented by arrow 209 extending from the block representing input power processing circuitry 203. Power conditioning provided by input power processing circuitry 203 may include rectification, such as half-wave or full-wave rectification, of an incoming AC electrical power. In various embodiments, power conditioning provided by input power processing circuitry 203 may include filtering, such as low pass, bandpass, or high pass filtering of the power being provided to the electrical components and devices included in control unit 201. In various embodiments, power conditioning provided by input power processing circuitry 203 may include changing a voltage level, a peak voltage level, or a peak-to-peak voltage level of the incoming electrical power relative to the power being provided by the input power processing circuitry to the electrical components and devices included in control unit 201. In various embodiments, power conditioning provided by input power processing circuitry 203 may include making power factor corrections and/or phase adjustments to the incoming electrical power relative to the power being provided by the input power processing circuitry to the electrical components and devices included in control unit 201.

In various embodiments, all or various combinations of these power conditioning processes may be performed by input power processing circuitry 203 on the power being provided by the input power processing circuitry to the electrical components and devices included in control unit 201. In one embodiment, the electrical power input provided to input power processing circuitry includes 120 VAC 60 Hz electrical power, and the output power provided by the input power processing circuitry 203 to the power-delivery circuitry 205 includes a rectified waveform. As further described below, an intermediate electrical waveform generated by the electrical waveform generator 204 and provided to the power-delivery circuitry 205 is used to switch ON and OFF, and otherwise control the coupling of the electrical power provided by the input power processing circuitry 203 to the electrodes of the element 250 through the electrical devices, such as switching devices, included in the power-delivery circuitry.

As shown in FIG. 2, electrical waveform generator 204 includes RF source 204A coupled to modulator 204B. RF source 204A may be configured to generate an electrical waveform having a frequency in a range of 10 kHz to 30 MHz, inclusive. Higher frequencies, for example frequencies up to and including 100 MHz, may be generated by the RF source 204A in various embodiments, and even higher frequencies, up to and including 300 gigahertz, may be generated by the RF source in other embodiments. RF source 204A is not limited to generating a waveform having any particular frequency. In some embodiments, RF source 204A generates an electrical waveform having a frequency of 6.78 Mhz. The frequency generated by RF source 204A may be set based on a determination in some embodiments with respect to the type of fluid, such a saline or water, and/or by the arrangement of the electrodes, such as electrodes 255 and return electrode 256, that the control unit is being configured to heat using non-contact RF heating. Further, the shape and the configuration of the waveform generated by RF source is not limited to any particular shape, and in some embodiments is a sine wave or similar shaped waveform. However, the shape and configuration of the electrical waveform generated by RF source 204A is not limited to a sine wave or similar shaped waveform, and may comprise a square wave, a sawtooth shaped waveform, a triangular shaped waveform, or any other waveform that provides a varying voltage over time.

The type of circuitry utilized by RF source 204A to generate the electrical waveform is not limited to any particular type of circuitry or to any particular technique for generating an electrical waveform. In some embodiments, RF source 204A includes one or more high speed timers configured to generate an varying voltage output signal. In various embodiments, RF source 204A includes a voltage controlled oscillator, or some other type of oscillator, configured to generate a varying voltage output signal. Other types of circuitry and techniques may be utilized as part of RF source 204A to generate the electrical waveform having a varying voltage output, and are contemplated for use as embodiment(s) of the RF source included in control unit 201.

As shown in FIG. 2, an output of RF source 204A is coupled to modulator 204B. Modulator 204B is configured to receive the electrical waveform generated by RF source 204A, and to modulate the electrical waveform to controllably generate an intermediate electrical waveform based on the electrical waveform received from the RF source. In various embodiments, modulator 204B is configured to produce a waveform by switching ON and switching OFF the electrical waveform received from the RF source 204A to produce a pulsed output waveform as the intermediate electrical waveform that is output from the modulator. The pulsed output waveform may comprise cycles having an overall time period including a first time period where the electrical waveform received from the RF source is switched ON, and a second time period following the first time period wherein the RF electrical waveform from the RF source is switched OFF. The overall time period for each cycle of the pulsed output waveform is not limited to a particular time period, and may be 8.3 milliseconds, or a time period less than or greater than 8.3 milliseconds for example in a range of 1 to 100 milliseconds, inclusive. In some embodiments, the duty cycle of the pulsed output waveform may be varied over a range from zero to one hundred percent, and in some embodiments may a duty cycle of fifty percent. In various embodiments the timing of the switching from an ON state to an OFF state and/or from the OFF state to the ON state corresponds with a zero-crossing voltage level of the electrical power being provided to the power-delivery circuitry by the input power processing circuitry 203. Using switching timing corresponding to the zero-crossing voltage level may reduce stressed on the switching devices including in the power-delivery circuitry 205, and may help reduce or eliminate issues related to power factor correction and the incoming electrical power being provided to the control unit 201 by power lines 202. Variations in time period, the duty cycle, or both the time period and the duty cycle of the pulsed output waveform that may be generated as an output from modulator 204B may be controlled and varied in order to control the overall amount of electrical energy that is to be delivered to the electrodes of an non-contact RF heating element, such as element 250, that is being regulated by the intermediate electrical waveform as further described below.

In addition to or instead of controlling the frequency of the electrical waveform provided by RF source 204A, modulator 204B may be configured to variably control a maximum voltage level or a voltage range, such as peak-to-peak voltage, of the electrical waveform received from the RF source. For example, modulator 204B may variably increase or decrease the amount of voltage variation, including varying a maximum voltage level or varying a voltage range (peak-to-peak voltage) of the electrical waveform received by the modulator from RF source 204A. The variations in the voltage level(s) generated by modulator 204B may then be provided as the intermediate electrical waveform that is output from the modulator. Controlling variations in the voltage levels of the intermediate electrical waveform output by modulator may be used to control the overall amount of electrical energy that is to be delivered to the electrodes of a non-contact RF heating element, such as element 250, that is being regulated by the intermediate electrical waveform as further described below.

In some embodiments, modulator 204B may be configured to modulate the electrical waveform received from the RF source 204A by varying the frequency of the electrical waveform to generate the intermediate electrical waveform that is then provided as an output from the modulator. Controlling variations in the frequency of the intermediate electrical waveform that is being output by modulator may be used to control the overall amount of electrical energy that is delivered to the electrodes of an non-contact RF heating element, such as element 250, that is being regulated by the intermediate electrical waveform as further described below.

As shown in FIG. 2, an output from modulator 204B is coupled to an input of power-delivery circuitry 205. In addition, electrical power output lines 220 are provided as electrical outputs from input power processing circuitry 203 and are coupled to power-delivery circuitry 205. Electrical power output lines 220 are configured to couple an electrical power source, for example which has been processed and provided by input power processing circuitry 203, to power-delivery circuitry 205. In various embodiments, the electrical power provided by power output line 220 is controllably output by power-delivery circuitry 205 based on and controlled by the intermediate electrical waveform received by the power-delivery circuitry from modulator 204B. In various embodiments, power-delivery circuitry 205 includes one or more electrical switching devices, such as a field-effect transistor (FET), such as but not limited to gallium nitride (GaN) devices, and/or metal-oxide-semiconductor field-effect transistors (MOSFET), such as but not limited to Silicon Carbide (SiC) or silicon MOSFETS. These devices may be configured to act as a switching devices to switch ON and thus couple electrical power provided by power lines 220 to the power-delivery circuitry to the outputs of the power-delivery circuitry coupled to electrode output terminal 206 (OUT 1) and the electrode return terminal 207.

The switching devices included in the power-delivery circuitry 205 are also configured to be controllably switched OFF, and thus to disconnect the electrical power being provided by power lines 220 to the power-delivery circuitry from the outputs of the power-delivery circuitry coupled to electrode output terminal 206 (OUT 1) and the electrode return terminal 207. In various embodiments, during the periods of time when the switching devices are switched ON, the switching devices included in the power-delivery circuitry 205 may be further controlled by the intermediate electrical waveform received from the electrical waveform generator 204 to vary for example the voltage level being provided at the electrode output terminal coupled to the switching device(s) in order to provide a varying voltage output waveform having variations corresponding to the variations of the intermediate electrical waveform to the electrodes of element 250. As further described below, the various parameters of the intermediate electrical waveform generated by the electrical waveform generator 204 may be controlled by input signals provided to the electrical waveform generator by control circuitry 210. In various embodiments, electrical waveform outputs provided to the electrodes of element 250 as an output from the power-delivery circuitry 205 and as controlled by the intermediate electrical waveform generated by electrical waveform generator 204 may be configured to produce non-contact radio-frequency heating of a fluid flowing through passageway 252 of the element.

As shown in FIG. 2, control circuitry 210 may include a computer system, such as a microprocessor and associated computer circuitry, that may include computer memory coupled to one or more computer processors, illustratively represented in FIG. 2 as memory 212 and processor 211, respectively. Memory 212 may store instructions and one or more parameter values that processor 211 may operate on to control the operation of control unit 201. For example, memory 212 may store one or more values corresponding to desired temperature outputs or to an acceptable range of temperature outputs for the heated fluid flow exiting the element 250. Processor 211 may use this desired temperate value, or the acceptable temperature range of values, to determine how to control the output of electrical energy provided at electrode output terminal 206 in order to control the heating of the fluid flow through element 250. Processor 211 may use inputs provided to control unit 201, such as temperature sensor signals provided by one or more temperature sensors 257, flow sensor inputs provided by flow sensor 259, ambient temperature inputs provided by ambient temperature sensor 264, and/or other inputs or parameters values for use in various algorithms used to regulate the generation of the intermediate electrical waveform, which in turn is used to control the power-delivery circuitry to provide electrical output waveforms to be provided to electrode output terminal 206, and thus used to regulate the heating of the fluid flow through passageway 252 and element 250 in a desired manner.

In various embodiments, control circuitry 210 may monitor, using one or more electrical sensors including in the input power processing circuitry 203 and/or the power-delivery circuitry 205, an impedance level of the electrical circuit provided across electrode 255 and return electrode 256. For example, the type of fluid flowing through passageway 252 may affect the impedance value of the electrical circuit that includes the area between electrode 255 and return electrode 256. By way of example, a fluid such as saline will present a different level of electrical impedance for an electromagnetic field established in the fluid as compared to water. In various embodiments, control circuitry 210 may be configured to measure an impedance that is presented in the electrical circuitry including the area between electrode 255 and return electrode 256, and to adjust the control parameters applied to the electrical waveform generator 204 and/or to the power-delivery circuitry 205 based on the sensed impedance measurements. In various embodiments, control circuitry 210 may be configured to estimate a temperature of a fluid flowing through or contained within passageway 252 of element 250 based on measurement(s) of the impedance of the circuit that includes the fluid present between electrode 255 and return electrode 256. By way of example, a measurement of the voltage, current, and phase applied to the electrodes 255, 256 may be made, and an impedance value (real and imaginary: R+jX), is determined based on these measured values. The determined impedance value is then comparted to values either in a look-up table, or applied to an equation in order to determined temperature value corresponding to the impedance value. In some embodiments, power and other variables, such as input voltage, may be used to determine a value for liquid conductivity, and then correlated to a temperature value of the liquid.

Control circuitry 210 may utilize one or more techniques to control the overall level of electrical energy provided to the electrodes of a non-contact radio-frequency heating element, such as element 250, and thus control the heating of a fluid flowing through the heating element. In various embodiments, control circuitry 210 may provide one or more control signals to input power processing circuitry 203. These control signals may allow the control circuitry to modify one or more parameters of the power that is to be or is being provided by the input power processing circuitry to the power-delivery circuitry 205. In various embodiments, control circuitry 210 may provide one or more control signals to electrical waveform generator 204 configured to control and/or vary the frequency of the intermediate electrical waveform being provided as an output from the electrical waveform generator. Varying the frequency of the electrical waveform generator's intermediate electrical waveform may change the overall impedance of the circuit that includes a fluid flowing past and/or positioned between electrodes of a non-contact radio-frequency heating element, and thus control the overall heating of the fluid. In various embodiments, control circuitry 210 may provide one or more control signals to electrical waveform generator 204 that are configured to control and/or vary one or more of voltage levels, such as peak voltage and/or peak-to-peak voltage, of the electrical output waveform provided as an output from the power-delivery circuitry 205. Varying one or more voltage levels of the intermediate electrical waveform being provided as an output from the electrical waveform generator 204 may change the overall level of electrical power being delivered by the power-delivery circuitry 205 to the electrodes of a non-contact radio-frequency heating element, such as element 250, and thus control the overall heating of the fluid passing through the non-contact radio-frequency heating element.

In various embodiments, control circuitry 210 may provide one or more control signals to electrical waveform generator 204, for example to modulator 204B, that are configured to control and/or generate a pulsed output of the intermediate electrical waveform provided as an output from electrical waveform generator 204 to the power-delivery circuitry 205, and thus control a duty cycle for the application of electrical power to the electrodes of a non-contact radio-frequency heating element, such as element 250. Controlling a duty cycle of the electrical power being provided as an output from the power-delivery circuitry 205 may change the overall level of electrical power being delivered to the electrodes of a non-contact radio-frequency heating element, and thus control the overall heating of the fluid passing through the non-contact radio-frequency heating element.

Various embodiments of control unit 201 include a user interface 214 communicatively coupled to control circuitry 210. User interface 214 may be configured to allow electrical communications, for example but not limited to communication utilizing a RS-232 format, between control circuitry 210 and one or more other computer systems, such as computer system 265 as illustrated in FIG. 2, which are external to control unit 201. In various embodiment, computer system 265 may be used to download programing and/or parameter values to control circuitry 210, which may then be stored in memory 212 and operated on by processor 211. Programing parameters may include information related to the type of non-contact radio-frequency heating element and/or the arrangement of electrodes that the control unit is being configured to be coupled to as part of a non-contact radio-frequency heating system, such as system 200. In various embodiments, parameters, such as a desired temperature or an acceptable temperature range of the output of heated fluid passing through a non-contact radio-frequency heating element that is electrically coupled to control unit 201 may be provided through user interface 214 to control circuitry 210. Other information, such as but not limited to the distance along a conduit extending from the output of the non-contact radio-frequency heating element to the point where the fluid is introduced into a patient may be provided to control circuitry 210 through user interface 214. Such information may be utilized by control circuitry 210 to determine the overall heating regiment that may be applied to heating a fluid flow that is passing through the element coupled to the control unit 201 by factoring in the amount of cooling that is likely to occur after the fluid exits the element and before introduction into the patient. Additional information that may be provided to control circuitry 210 through user interface 214 may include information related to the types and numbers of sensors included as part of a non-contact radio-frequency heating unit that the control unit 201 is to be coupled to, and the type of fluid that is being passed through the element for heating purposes. In various embodiments, user interface 214 may also be configured to output information from control circuitry 210 to the external computer systems that may be coupled to the user interface, such as temperature readings, temperate profiles related to a heating process performed by the control unit 201, and/or output of data related to the control parameters that were utilized by the control unit to produce these temperate reading and temperature profiles.

In various embodiments, control unit 201 may include a temperature output 216 that is electrically coupled to control circuitry 210. Temperature output 216 may provide an output signal, such as a voltage output, that is indicative of a current temperature value for a fluid that is being heated by or at least flowing through the non-contact radio-frequency heating element coupled to control unit 201. The temperature output signal may in some embodiments be provided to a display device configured to visually display a value corresponding to the temperature indicated by the signal provided at the temperature output 216.

Control unit 201 may provide various features and perform various functions related to safety and regulation of a non-contact radio-frequency heating system such as system 200. For example, various types of shielding may be provided to limit or eliminate electromagnetic radiation associated with the higher frequencies that may be generated by and transmitted through the system. In various embodiments, certain fault conditions may be monitored for, and when detected may result in a shutdown and/or a power down of one or more portions of the control unit. For example, an overvoltage and/or an over current condition occurring in the power input power processing circuitry, 203, electrical waveform generator 204, and/or power-delivery circuitry 205 may be monitored for, and if any voltage or current levels exceed acceptable levels, one or all of these portions of the control unit 201 may be powered down. In various embodiments, the temperature of one or more switching devices, such as MOSFETs, that may be included in power-delivery circuitry 205 may be monitored, and if these temperature(s) exceed acceptable limits, the power-delivery circuitry 205 may be powered down. In various embodiments, a parameter related to a maximum fluid temperature sensed by one or more temperature sensors sensing temperatures of the fluid at or passing through the non-contact radio-frequency heating element coupled to the control unit may be monitored, and if the fluid temperature(s) exceeds any threshold level(s) set for fluid temperature, the control unit may shut down the electrical waveform generator and/or power-delivery circuitry of the control unit so that the electrical output waveform is disconnected from the electrode output terminal(s) of the control unit and is no longer being applied to the electrodes of the non-contact radio-frequency heating element. In various embodiments, a flow level or volume of fluid flow passing through the non-contact radio-frequency heating element is monitored, and if no flow is detected, or for example a minimum level of fluid flow is not detected, the control unit may be configured to stop providing electrical energy to the electrodes of the non-contact radio-frequency heating element, and thus cease any further heating of the fluid until and/or unless a fluid flow is detected, or the minimum level of fluid flow is re-established through the non-contact radio-frequency heating element.

In various embodiments the control circuitry 210 performs the monitoring and alarm function, and controls output signals to the electrical waveform generator 204 and/or the power-delivery circuitry 205 to power down or shut down portions of the control unit when an unacceptable, fault, or alarm condition is detected. In various embodiments, other devices, such as fuses and/or circuit breaker, which may or may not be controlled by the control circuitry 210, may provide protection, such as protection against electrical overloads within the control unit 201 and/or associated with the electrical power being provided to the non-contact radio-frequency heating element by the control unit and/or to the control unit from any electrical power input sources coupled to lines 202.

The overall wattage level of electrical energy provided by control unit 201 to a RF heating element, such as element 250, is not limited to any particular wattage, and in various embodiments is configured and controlled based on the particular application, such as the type of fluid being processed, the amount of heating of the fluid that is required, and/or the configuration of the RF heating element itself. In various embodiments, a control unit, such as control unit 250, is configured to provide an overall wattage level in a range of 0 to 500 watts of electrical power in a controlled manner to a RE heating element. Embodiments may include higher wattage levels for example up to and including 2000 watts or more, again depending on the application. In various arrangements, the application of the electrical energy to the fluid as part of the RF heating process may generate bubbles, such as gas bubbles, in the fluid. The formation of bubbles may create issues, for example in medical applications involving a patient, and/or may change the impedance across the electrodes of the RF heating element, which in turn may affect the regulation of the RF heating process being performed by the control unit and the RF heating element. In various embodiments, operations utilizing the RF heating element may include positioning the exit end of the element in a vertical or upward orientation to: 1) allow for all bubbles to exit the tubing, 2) for preventing any new bubbles from getting trapped, 3) and/or allow any generated gas to escape. In various embodiments, one or more bubble sensors may be incorporated into a RF heating system, such as system 200, to detect the presence of gas bubbles in the fluid being heated, and to provide an output signal to the control unit 201 indicative of the presence or absence of bubbles that may be detected in the fluid. An embodiment of a bubble sensor may comprise a light source, such as but not limited to a laser light source, and a photo detector, such as but not limited to a photodiode, configured to detect the light provided by the light source. The bubble detector may be configured to provide an output signal that is indicative of the presence or absence of bubbles in the fluid. In various embodiments, the bubble sensor may be built into the RF heating element, and/or may be incorporated into the fluid output conduit, such as fluid output conduit 254 as shown in FIG. 2, for example as sensor 259 as shown in FIG. 2. In various embodiments, the output signal from the bubble sensor may be received by control circuitry included in the control unit, such as control circuitry 210, and used to regulate the level of electrical energy being applied to the fluid that is flowing through or is contained within the RF heating elements. In various embodiments, an output signal from the bubble sensor may be processed by the control circuitry, causing the control circuitry to reduce the level of electrical energy being provided to the RF heating element, and thus reduce or eliminate the formation of bubbles in the fluid. In various embodiments, the detection of bubbles in the fluid may be considered an alarm condition, and when bubbles are detected, for example based on the output signal generated by a bubble sensor, the control circuitry of the control unit may be configured to shut down or otherwise stop providing electrical energy to the RF heating element, and/or may output an alarm signal, for example to an external computer system such as computer system 265, intended to alert a system user, such as a medical technician or operator, of the detection of the bubbles in the fluid being processed by the RF heating element.

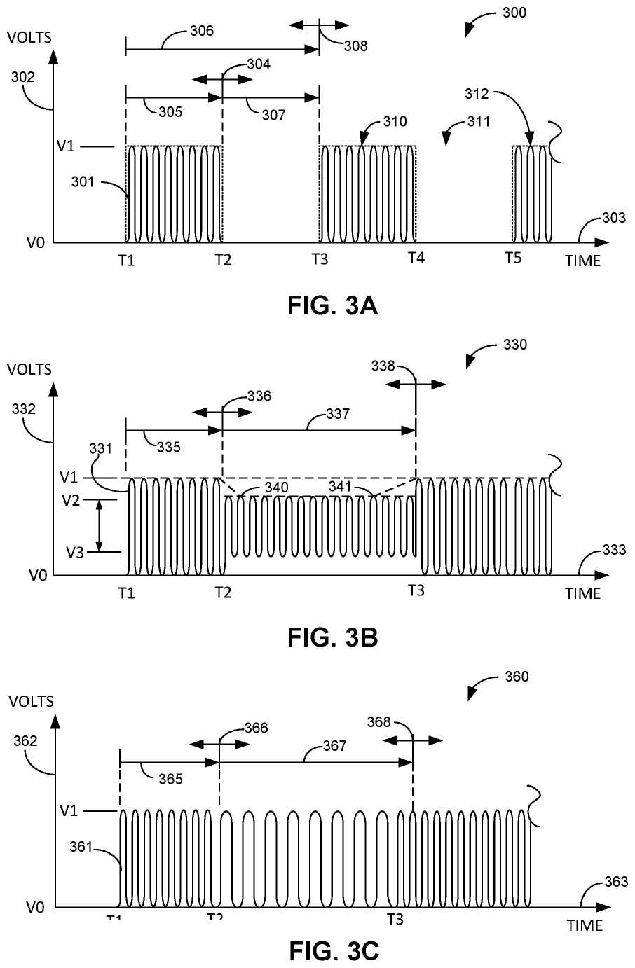

FIGS. 3A-3C illustrate graphs 300, 330, and 360, respectively, of various electrical output waveforms that may be generated and applied to one or more electrodes of a non-contact radio-frequency heating element according to at least one embodiment. The variations in the waveforms illustrated by each of graphs 300, 330, and 360, alone or in some combination, may be used to control the electrical power delivered by a control unit, such as control unit 201 (FIG. 2), and thus provide control over the heating of a fluid that is flowing through or that is contained within a non-contact radio-frequency heating element, such as element 250 (FIG. 2) that is coupled to receive the electrical power provided by the control unit.

FIG. 3A illustrates a graph 300 of an electrical output waveform 301 that may be generated and applied to one or more electrodes of a non-contact radio-frequency heating element according to at least one embodiment. Graph 300 includes a vertical axis 302 representing voltage levels, and a horizontal axis 303 representing time. Waveform 301 as illustrated in FIG. 3A is a sine wave having a varying voltage level extending between voltage level V0 and voltage level V1 at some predetermined frequency. In some embodiments, the frequency of waveform 301 is 6.78 MHz. However, the frequency of waveform 301 is not limited to 6.78 MHz, or to a particular frequency, and in various embodiments may be any frequency in a range of 10 kHz to 30 MHz, inclusive. Other embodiments of waveform 301 may be as high as 100 MHz, or up to and including 300 GHz. Further, waveform 301 is not limited to a waveform comprising a sine wave, and in various embodiments may be a waveform that is not a sine wave, for example a square wave, a sawtooth shaped waveform, or a triangular shaped waveform.

As shown in FIG. 3A, prior to time T1, waveform 301 is maintained at voltage level V0, but is turned ON at time T1, and remains in an ON state over the time period represented by arrow 305 until time T2. At time T2, waveform 301 is switched to an OFF state, and remains at the V0 voltage level over a second time period represented by arrow 307 that begins at time T2 and ends at time T3. The combination of the first time period 305 and the second time period 307 extends from time T1 to time T3, and is represented by time period illustrated by arrow 306. The time period represented by arrow 306 represents the time period for one ON/OFF cycle of waveform 301, wherein during the first time period 305 waveform 301 oscillates at a predefined frequency, and during the second time period 307 waveform 301 is held at a constant voltage level represented by voltage V0. As such, the relative length of the first time period compared to the relative time period represented by the second time period (arrow 307) represents a duty cycle for the ON/OFF switching of waveform 301 over period 306. In various embodiments, the peak-to-peak voltage value for waveform 301 may include a range of 5 to 20,000 volts, inclusive.

Following time T3, a subsequent time period 310 may include waveform 301 switched to an ON state, extending to time T4 as represented by arrow 310, wherein at time T4 waveform 301 is switched back to the OFF state for a time period represented by arrow 311 extending from time T4 to time T5. The time periods 310 and 311 represent another and subsequent ON/OFF switching cycle of waveform 301 having a duty cycle and an overall period that may be adjusted to control the overall amount of electrical power provided during this subsequent cycling of waveform 301. Additional switching cycles, as represented by the partially illustrated time period of at arrow 312, may follow after time T5 and may include variable time periods and/or variable duty cycles as described above for the previous ON/OFF switching cycles of waveform 301.

The ON/OFF switching of waveform 301 may represent a switching of an electrical power output from an electrical waveform generator (e.g., electrical waveform generator 204, FIG. 2), that is then applied to a power-delivery circuitry, such as power-delivery circuitry 205 (FIG. 2). Controlling the switching on and off of the power-delivery circuitry (e.g., power-delivery circuitry 205, FIG. 2) may result in delivery of a set of ON/OFF pulses of electrical power provided for example by input power processing circuitry (203—FIG. 2) in the form of electrical waveform corresponding to waveform 301 to one or more electrodes of a non-contact radio-frequency heating element to control heating of a fluid flowing through or contained within the non-contact radio-frequency heating element. As shown in FIG. 3A, the overall time included in time period 306 may be varied, and represented by the double arrows 308 coupled to line at time T3, to increase or decrease the rate at which the ON/OFF cycles are provided to the electrodes. In addition, the duty cycle as shown in FIG. 3A is represented a being a fifty-percent duty cycle, with the first time period (arrow 305) having an equal time span as the second time period (arrow 307), so that the waveform is providing a varying voltage for half the time period 306, and is providing no voltage level during the second half of time period 306. However, as represented by the double arrows 304 coupled to the line at time T2, the relative time spans of the first time period and the second time period may be varied in order to change the duty cycle of the waveform 301. Increasing the duty cycle, that is, extending the first time period relative to the second time period, would increase the relative time during time period 306 when waveform 301 is providing electrical power, and decreasing the duty cycle would decrease the relative time period 306 during which waveform 301 is providing electrical power. By adjusting either the period 306, the duty cycle of period 306, or both the period of 306 and the duty cycle of waveform 301, control over the amount of electrical power, and thus over the amount of heating of a fluid flowing through or contained within a non-contact radio-frequency heating element receiving the electrical power provided by waveform 301 may be controlled.

FIG. 3B illustrates a graph 330 of an electrical output waveform 331 that may be generated and applied to one or more electrodes of a non-contact radio-frequency heating element according to at least one embodiment. Graph 330 includes a vertical axis 332 representing voltage levels, and a horizontal axis 333 representing time. Waveform 331 as illustrated in FIG. 3B is a sine wave having a varying voltage level extending between voltage level V0 and voltage level V1 at some predetermined frequency over a first time period 335 extending from time T1 to time T2, and a varying voltage level extending between voltage level V2 and voltage level V3 over a second time period 337 extending from time T2 to time T3. In some embodiments, the frequency of waveform 331 is 6.78 MHz. However, the frequency of waveform 331 is not limited to 6.78 MHz, or to a particular frequency, and in various embodiments may be any frequency in a range of 10 kHz to 30 MHz, inclusive, or in some embodiments up to 100 MHz and in still other embodiments up to 300 GHz. Further, waveform 331 is not limited to a waveform comprising a sine wave, and in various embodiments may be a waveform that is not a sine wave, for example a square wave, a sawtooth shaped waveform, or a triangular shaped waveform.

As shown in FIG. 3B, the variations in the peak-to-peak voltage levels of waveform 331 during time period 335 is larger than the variations in the peak-to-peak voltage levels for waveform 331 during time period 337. In various embodiments, waveform 331 is an intermediate electrical waveform generated by and electrical waveform generator, such as electrical waveform generator 204 (FIG. 2) and is used to control the power-delivery circuitry that is electrically coupled to the electrodes of a heating element, such as element 250 (FIG. 2), by controlling the power-delivery circuitry to provide and electrical power to the electrodes having a waveform that corresponds to waveform 331. As such, during time period 335 waveform 331 will deliver more electrical power on average for a given period of time compared to amount of electrical power delivered on average for a same given period of time while providing the variation in waveform 331 as illustrated for time period 337. By controlling the overall peak-to-peak voltage level of waveform 331, control over the amount of electrical power, and thus over the amount of heating of a fluid flowing through or contained within a non-contact radio-frequency heating element receiving the electrical power provided by waveform 331 may be controlled. As shown in graph 330, the point in time where the voltage variation is changed at time T2 can be varied back or forward relative to time axis 333, thus switching the voltage variation represented by time period 337 to an earlier or a later time. Similarly, the time T3 where the voltage variation of waveform 331 is again switched to a different level for the peak-to-peak voltage may be varied, as illustrated by arrows 338, relative to time axis 333.

As further illustrated in FIG. 3B, at time T3 the peak-to-peak voltage variation of waveform 331 returns to a level extending between V0 and V1, which comprises a higher peak-to-peak voltage value for waveform 331 compared to the peak-to-peak voltage variations of waveform 331 during time period 337. Thus, waveform 331 provides more electrical power, and thus generates a greater amount of heating of a fluid flowing through or contained within a non-contact radio-frequency heating element compared to the electrical power and heating generated by waveform 331 for a same period of time during time period 337. The time for the change in the variation of the voltage levels between time period 335 and 337 may be configured as a ramp up or a ramp down relative to peak-to-peak voltage levels, as represented by dashed ramp lines 340 and 341. Further, the variation in peak-to-peak voltage levels is not limited to the use of two different voltage levels, and may include use of any number of discrete voltage levels, or variation of the peak-to-peak voltage level over a continuous range of values for varying the voltage levels. In various embodiments, the peak-to-peak voltage values for waveform 331 may vary from over a range of 5 to 20,000 volts, inclusive. In addition to varying peak-to-peak voltage, and output waveform 331 may be switched ON and OFF in a manner similar to that described above for waveform 301 and graph 300.

FIG. 3C illustrates a graph 360 of an electrical output waveform 361 that may be generated and applied to one or more electrodes of a non-contact radio-frequency heating element according to at least one embodiment. Graph 360 includes a vertical axis 362 representing voltage levels, and a horizontal axis 363 representing time. Waveform 361 as illustrated in FIG. 3C is a sine wave having a varying voltage level extending between voltage level V0 and voltage level V1 at some predetermined frequency over a first time period 365 extending from time T1 to time T2, and a varying voltage level having a different frequency and extending between voltage level V0 and voltage level V1 over a second time period 367 extending from time T2 to time T3. In various embodiments, the peak-to-peak voltage values for waveform 361 may vary from over a range of 5 to 20,000 volts, inclusive. In some embodiments, at least one of the frequencies represented by waveform 361 over one of time period 365 or 367 is a frequency of 6.78 MHz. However, the frequency of waveform 361 is not limited to 6.78 MHz, or to a particular frequency, and in various embodiments may be any frequency in a range of 10 kHz to 30 MHz, inclusive. Further, waveform 361 is not limited to a waveform comprising a sine wave, and in various embodiments may be a waveform that is not a sine wave, for example a square wave, a sawtooth shaped waveform, or a triangular shaped waveform.

As shown in FIG. 3C, waveform 361 oscillates at a first frequency over time period 365, and then oscillates at a different, lower frequency over time period 367. After time T3, waveform 361 returns to having a frequency the same as the frequency of waveform 361 over time period 365. By varying the frequency of waveform 361, the impedance of the circuit including electrodes and the fluid passing through or contained within a non-contact radio-frequency heating element receiving the electrical power in the form of waveform 361 varies, and thus the total amount of electrical power, and therefore the heating of the fluid may be varied and controlled by the variation of the frequency of waveform 361. For example, in various embodiments waveform 361 is an intermediate electrical waveform generated by and electrical waveform generator, such as electrical waveform generator 204 (FIG. 2), and is used to control the power-delivery circuitry, such as power-delivery circuitry 205 (FIG. 2) that is electrically coupled to the electrodes of a heating element, such as element 250 (FIG. 2) by controlling the power-delivery circuitry to provide an electrical power to the electrodes having a waveform that corresponds to waveform 361. The range of frequency over with the frequency of waveform 361 may be varied is not limited to any particular frequency or range of frequencies and various embodiments includes varying the frequency over a range of frequencies extending from 10 kHz to 30 MHz, inclusive, or in some embodiments up to 100 MHz and in still other embodiments up to 300 GHz.

In various embodiments, the time period during which waveform 361 is provided as having a first frequency illustrated by arrow 365 may be varied, as illustratively indicated by double arrows 366, and/or the time period during which waveform 361 is provided as having a second frequency different from the first frequency, as illustrated by arrow 367 may be varied, as illustratively indicated by double arrows 368. In addition to varying frequency of waveform 361 over different and subsequent time periods, waveform 361 may be switched ON and OFF in a manner similar to that described above for waveform 301 and graph 300. In the alternative or in addition to switching the waveform 361 ON and OFF, the overall peak-to-peak volte of waveform 361 may be varied in a same or similar manner as described above with respect to graph 330 and waveform 331.

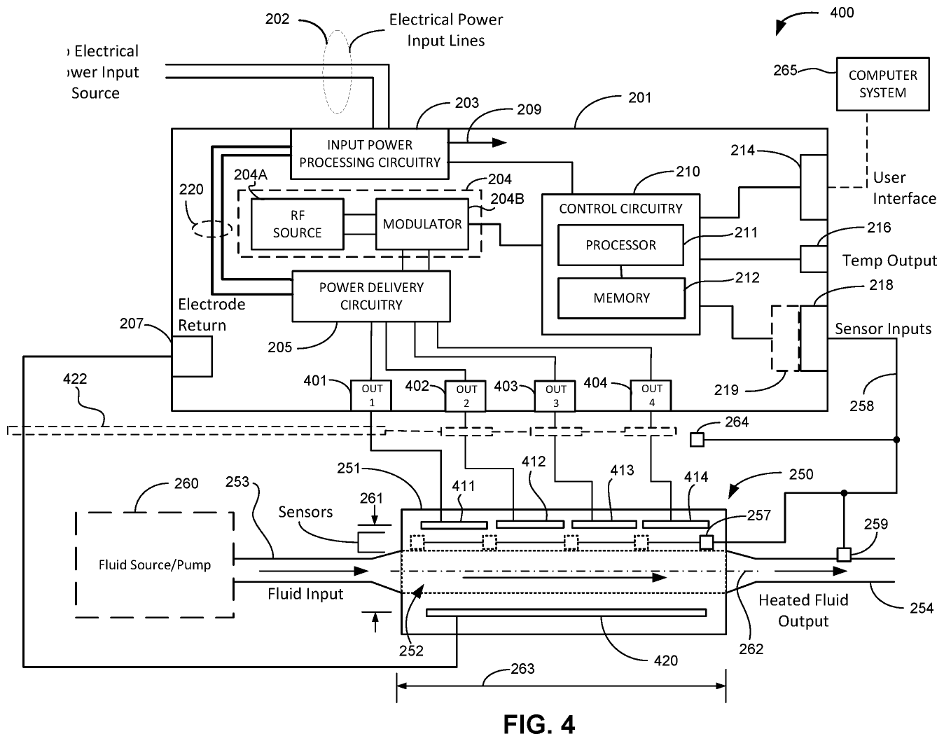

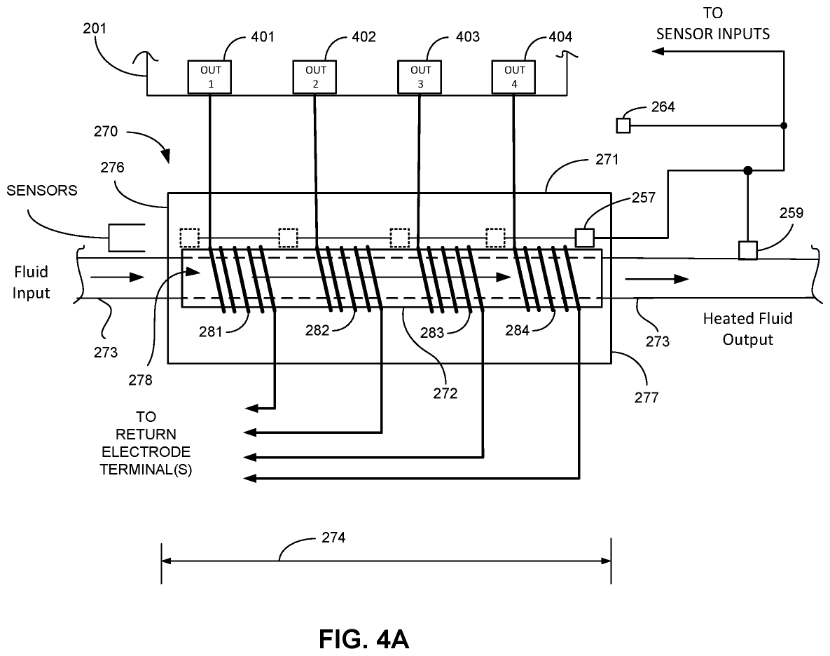

FIG. 4 illustrates a schematic block diagram of a fluid non-contact radio-frequency heating system 400 (hereinafter “system 400”) according to at least one embodiment. As shown in FIG. 4, system 400 includes many of the same devices and electrical circuitry, including a non-contact radio-frequency heating element 250 that is electrically coupled to a non-contact radio-frequency heating control unit 201 (hereinafter “control unit 201”). System 400 may be configured to provide a controlled level of non-contact radio-frequency heating to a flow of fluid passing through element 250 by non-contact radio-frequency heating of the fluid flow using electrical energy provided and controlled by control unit 201 to one or more electrodes included in element 250, as described above with respect to FIG. 2 and system 200. Therefore, the same references numbers are used in FIG. 4 to refer to the same or similar devices as illustrated in FIG. 2 with reference to system 200, with variations and differences between the two systems further described below.

As shown in FIG. 4 and for system 400, control unit 201 includes four separate electrode output terminals, including output 1 (401), output 2 (402), output 3 (403), and output 4 (404). Each of the electrode output terminals is coupled to power-delivery circuitry 205, and is configured to receive an electrical output waveform provided to the electrode output terminal from the power-delivery circuitry. In addition, each of the electrode output terminal 401, 402, 403, and 404 is coupled to a respective one of the separate electrodes 411, 412, 413, and 414 included in body 251 of non-contact radio-frequency heating element 250. As shown in FIG. 4, electrode output terminal 401 is coupled to electrode 411, electrode output terminal 402 is coupled to electrode 412, electrode output terminal 403 is coupled to electrode 413, and electrode output terminal 404 is coupled to electrode 414. Each of these electrodes individually or together, in combination with return electrode 420, may be referred to as a set of electrodes.

In various embodiments, each of electrodes 411, 412, 413, and 414 is electrically isolated from one another, and positioned above and proximate to passageway 252 of non-contact radio-frequency heating element 250. A return electrode 420 is electrically isolated from each of the electrodes 411, 412, 413, and 414, and is positioned below passageway 252 on an opposite side of the passageway relative to electrodes 411, 412, 413, and 414. As shown in FIG. 4, each electrode 411, 412, 413, and 414 extends parallel to longitudinal axis 262, and along a portion of the length dimension 263 of the element 250 that is different from the portion of the length dimension 263 over which any of the other electrodes extend. Return electrode 420 may extend parallel to electrodes 411, 412, 413, and 414, and extend over a length dimension along the longitudinal axis 262 that includes all of the length dimension extended over by each of the electrodes 411, 412, 413, and 414.

In various embodiments, electrode conductor wiring 422 may include shielding coupled to return electrode 420, and to electrode return terminal 207 of control unit 201, wherein separate sets of wiring may be utilized to couple and/or shield each individual electrode 411, 412, 413, and 414 along with a respective return conductors for coupling the respective electrode and return electrode 420 to control unit 201. In various embodiments, instead of being formed as a single electrode, return electrode 420 may comprise individual electrodes (not specifically shown in FIG. 4), each of the individual return electrodes positioned opposite a respective one of electrodes 411, 412, 413, and 414, thus forming four sets of individual electrode/return electrode pairs. Each of electrodes 411, 412, 413, and 414, along with an individual return electrode, may be referred to as a set of electrodes.

In various embodiments, electrodes 411, 412, 413, and 414, along with return electrode 420, are generally formed having a planar flat shape. However, embodiments of the electrodes and the return electrode or return electrodes are not limited to having a planar flat shape, and may for example have a curved arch-shape the extends at least partially around the longitudinal axis 262 at some radial distance from the longitudinal axis while remaining electrically isolated from direct contact with all other electrodes included in the element 250.

In various embodiments, control unit 201 may be configured to individually control an electrical output waveform provided to each of the electrode output terminals, 401, 402, 403, and 404, thus providing individually controlled outputs to each of the electrodes 411, 412, 413, and 414, respectively. In various embodiments, control unit 201 may operate all of the electrode output terminals 401 at the same time with respect to a switched ON and OFF state for application of an electrical output waveforms to the electrodes. In various embodiments, control unit 201 or may operate these ON and OFF states to individually control the output of an electrical output waveform to the respective electrode output terminals, and thus to the electrodes of the element 250 on an individual basis, wherein one or more of the electrode output terminals may be switched to an OFF state while other ones of the electrode output terminals are switched to an ON state. In various embodiments, an added number of temperature sensors, for example five temperature sensors as illustrated in FIG. 4, may be included in the non-contact radio-frequency heating element 250 and configured to generate sensor output signals related to sensed temperatures at or proximate to each of the electrodes. The sensor output signals from the temperature sensors are coupled to the control unit 201 through sensor input 218 to allow the control unit 201 to determine temperature gradients that may exist over the length of the element 250, and thereby provide more resolution with respect to heating control applied through the electrical output waveforms being applied to the individual electrodes 411, 412, 413, and 414.

In various embodiments, different electrical output waveforms, such as but not limited to the electrical output waveforms described above with respect to FIGS. 3A-3C, may be applied to one or more of the electrode output terminals 401, 402, 403, and 404 at any given time to control the heating of a fluid passing through or contained within passageway 252. For example, electrode output terminal 401 may receive an electrical output waveform continuously, wherein one or more of electrode output terminals 402, 403, and/or 404 may receive a pules electrical output waveform such as waveform 301 as illustrated and described with respect to FIG. 3A. Varying the waveforms, and thus the amount of heating provided by the electrodes at different positions relative to the length dimension 263 of the element 250 may provide a more uniform heating in a smaller overall length dimension for element 250 compared to a single electrode embodiment of the element. Other variations of the control scheme for multiple electrodes provided in a non-contact radio-frequency heating element are possible, and are contemplated for use by system 400 as illustrated and described with respect to FIG. 4. Further, embodiments of system 400 are not limited to having a particular number of electrode output terminals for controlling electrodes, such as the four electrode output terminals as illustrated in FIG. 4, and may include embodiments that comprise less electrodes, such as two or three electrode terminal outputs, or more electrode terminal outputs, such as five or more electrode terminal outputs, that may be configured to control multiple electrode or multiple electrode sets provided within or as part of an electric heating element configured to be electrically coupled to the control unit.

FIG. 4A illustrates a schematic block diagram including a non-contact radio-frequency heating element 270 according to at least one embodiment. As shown in FIG. 4A, non-contact radio-frequency (RF) heating element 270, (hereinafter “element 270), includes a heating element body 271 (hereinafter “body 271”), having an outer tube 272 extending through at least a portion of body 271, and an inner tube 273 that is at least partially encircled by outer tube 272. Inner tube 273 extends through both the outer tube 272 and body 271. Inner tube 273 extends through a first end 276 of body 271, through the body along a length dimension 274 of the body, and out of a second end 277 of the body that is opposite first end 276. Inner tube 273 is configured to provide a passageway 278 for a flow of fluid through body 271. In various embodiments, inner tube 273 is formed from an electrically insulative material, such as a plastic material, although embodiments of the inner tube are not limited to any particular type of electrically insulative material. In various embodiments, outer tube 272 is formed from a material such as metal, stainless steel, or other metallic material that allows the inductive fields generated by the inductive coils 281, 282, 283, and 284 to be imposed on the area within inner tube 273, including passageway 278. However, embodiments of the material or type of materials that may be used to form outer tube 272 are not limited to a particular type of material or type of material, and any material or type of materials compatible with the operation of the inductive coils in heating a fluid that is flowing through or contained within passageway 278 may be used to form the outer tube. As further shown in FIG. 4A, a set of inductive coils 281, 282, 283, and 284 are wound around outer tube 272 and spaced, respectively, along the longitudinal dimension of the outer tube. Each of the inductive coils 281, 282, 283, and 284 are electrically coupled to a respective one of the electrode output terminals of control unit 201, and to the return electrode terminal(s) of control unit 201. As shown in FIG. 4A, inductive coil 281 is coupled to electrode output terminal 401 (OUT 1), inductive coil 282 is coupled to electrode output terminal 402 (OUT 2), inductive coil 283 is coupled to electrode output terminal 403 (OUT 3), and inductive coil 284 is coupled to electrode output terminal 404 (OUT 4) of control unit 201. Each of the inductive coils is also electrically coupled to one or separate ones of the return electrode terminals included as part of control unit 201.

The windings forming inductive coils 281, 282, 283, and 284 are not limited to any particular type of winding, or to any particular number of turns per used to form each coil, or to any particular type of material used to form the inductive coils. In some embodiments, each of inductive coils 281, 282, 283, and 284 comprises a same type of electrical conductor, such as a conductive metal such as copper, aluminum, silver, or gold, which may be used to form each winding, and a same number of turns of the electrical conductor. However, embodiments of the element 270 are not limited to having four coils in number, and may have more or less than four coils, including having just a single (one) coil. Further, embodiments of element 270 are not limited to having each of a plurality of coils included in the element comprising a same type of coil winding. For example, one or more of a plurality of coils included in element 270 may include more or less turns of winding of the electrical conductor used to form the inductive coil, and/or may be formed from a different electrical conductor, for example a different gauge of wire or other conductive element used to form the inductive coil(s).

In operation, control unit 201 may be configured to provide one or more electrical output waveform(s) to inductive coils 281, 282, 283, and 284 in order to generate an electromagnetic field in the area surrounding each inductive coil, including within the area surrounding each inductive coil included within passageway 278 of inner tube 273. The electromagnetic field(s) generated by inductive coils 281, 282, 283, and 284 may be configured produce heating for fluid that is flowing through or contained within the passageway 278. In various embodiments, control unit 201 applied a same electrical output waveform to each of inductive coils 281, 282, 283, and 284 at or over a same time period, including applying a pulsed electrical output waveform to each of the inductive coils 281, 282, 283, and 284 at a same period and same phase relative to the pulses of the electrical output waveform. However, embodiments may include control unit 201 providing different electrical waveform(s) to one or more of the inductive coils 281, 282, 283, and 284 at a same or at different times, wherein various combinations of the inductive coils 281, 282, 283, and 284 may be energized and de-energized at different time relative to one another and energized using different electrical output waveforms at a same or different time relative to the electrical waveform(s) being applied to energize other ones of the inductive coil. By varying and controlling the electrical waveform(s) used to energize the inductive coils 281, 282, 283, and 284, and/or the timing of the energization of each of the inductive coils 281, 282, 283, and 284, either individually or together is some combination, the heating of the fluid that is flowing through or contained within passageway 278 may be controlled.

Embodiments utilizing element 270 may be configured and operated to provide any of the features and to perform any of the functions related to heating, sterilization, or other processing of fluid as described throughout this disclosure, and any equivalents thereof. For example, as shown in FIG. 4A element 270 includes any combination of one or more temperatures sensors 257, one or more flow sensors 259, and/or one or more ambient temperature sensors 264. Output signals provided by these sensors, when present, may be coupled to control unit 201 and the corresponding information provided by the output signals incorporated into the temperature regulation being provided by controller 201 as described in various portions of the disclosure.

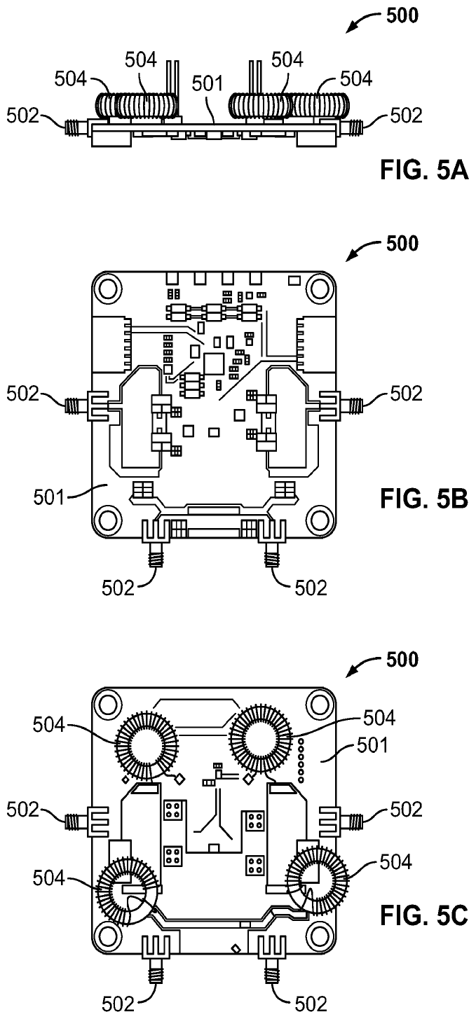

FIGS. 5A-5C illustrate side, top, and bottom views, respectively, of an electrical waveform generator/power-delivery circuitry that includes control circuits 500 according to at least one embodiment. As illustrated in the FIG. 5A, electrical waveform generator/power-delivery circuitry 500 includes a printed circuit board 501 with devices mounted on both the top and bottom sides of the board. In various embodiments, four separate electrode output terminals 502 are mounted to and extend from the side of the board 501. In various embodiments, four separate inductive coils 504 are mounted to the top surface of board 501. The inductive coils 504 may be configured to provide impedance matching, in conjunction with other electrical components, such as one or more capacitors (not shown in FIG. 5A) for the respective four electrode output terminals 502.

FIG. 5B illustrates a bottom side view of printed circuit board 501 and the relative locations of the individual electrode output terminals 502. FIG. 5C illustrates a top side view of printed circuit board 501 and the relative locations of the individual inductive coils 504. In various embodiments, printed circuit board 501 is square in shape and having a width and length dimension in a range of 3 to 5 inches, inclusive.

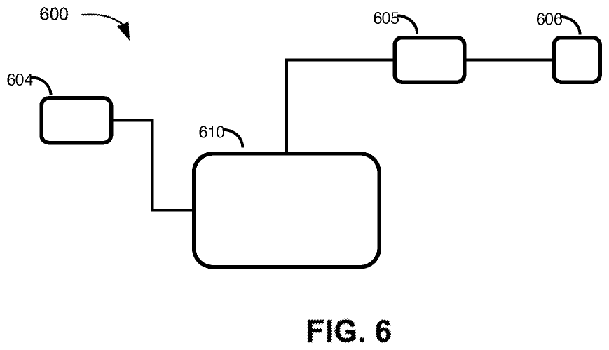

FIG. 6 illustrates an electrical schematic diagram 600 of an embodiment of electrical circuitry for a non-contact radio-frequency heating control unit according to at least one embodiment. In various embodiments, one or more portions of diagram 600 may represent the electrical circuitry and/or the circuit layout for a control unit, such a control unit 201, as illustrated and described with respect to FIG. 2 and FIG. 4. As shown in diagram 600, the electrical circuitry includes a temperature sensor amplifier 604, driver/switching circuitry 605, at least one electrode output terminal 606, and a microprocessor 610. Microprocessor 210 may be an example embodiment that includes functions, such as the functions provided by electrical waveform generator 204 (FIGS. 2 and 4), and may be configured to perform one or more of the functions and provide any combination of the features as described above with respect to control unit 201, and any equivalents thereof.

Driver/switching circuitry 605 may be an example embodiment of the power-delivery circuitry 205 (FIGS. 2 and 4), and may be configured to perform one or more of the functions and provide any combination of the features as described above with respect to control unit 201, and any equivalents thereof. Electrode output terminal 606 may be an example embodiment of any of the electrode output terminals (terminals 206, 207 in FIG. 2, terminals 401, 402, 403, 404, and 207 in FIG. 4), and may be configured to perform one or more of the functions and provide any combination of the features as described above with respect to control unit 201, and any equivalents thereof.

Microprocessor 610 as illustrated in FIG. 6 may be an example embodiment of some or all of the circuitry included in control circuitry 210 (FIGS. 2 and 4). A particular microprocessor, specifically a STMICROELECTRONICS® ARM® CORTEX®-M4 microcontroller, is illustrated in schematic diagram 600. However, microprocessor 610 is not limited to any particular type of microprocessor or microcontroller, and the particular microcontroller as illustrated in schematic diagram 600 is intended as a non-limiting and illustrative example of a type of microprocessor that may be used as part of the control circuitry included in a control unit such as control unit 201.

Various features and functions of the embodiment of a non-contact radio-frequency heating control unit have been described additional features and function that may be provided by and included as part of the embodiments of the non-contact radio-frequency heating control units as described throughout this disclosure are further described below.

Embodiment of the non-contact radio-frequency heating may be performed using frequencies in a range of 10 kHz to 30 MHz, or as high as 100 MHz, or as high as 300 GHz, which may allow a volume of liquid to be heated faster with a lower surface area to volume ratio as the energy is transferred into the liquid more uniformly. The energy may also be transferred into the liquid through a non-conductive surface to eliminate the risk of forming steam and/or bubbles due to “hot spots” generally accompanied with rapid heating using conductive methods. The end result is similar to microwave heating of a liquid except higher electrical to thermal efficiencies can be realized. Using a resonant inverter at megahertz frequencies also may provide very fast response time and fine control over the heating system. Strategies for passive/natural power factor correction may be incorporated that limit or eliminate the need for an active power factor correction stage common in more conventional switching regulators. In various embodiments, the control circuitry of the control unit may provide output signals to control a device, such as fluid pump, wherein the flow rate of the liquid is adjusted so as to maintain the monitored temperature at, or within a band around, a constant value.

In various embodiments, the passageway for the flow of fluid to be heated includes a flexible passageway. In various embodiments, the fluid to be heated is an ionic liquid. In various embodiments, the fluid to be heated is saline, and is physiological saline. In various embodiments, wherein the temperature of the fluid being heated is to be maintained within a temperature range of between 49° C. and 51° C., inclusive. In various embodiments, the fluid exiting the conduit conveying the heated fluid from the non-contact radio-frequency heating element is configured for conveying heat to a liquid and delivering said liquid at a temperature elevated above that of the human body into an external body orifice, such as but not limited to a urinary meatus of a patient. Various embodiments further include a catheter extending through the urethra of a patient for receipt within the bladder and configured to convey a fluid heated by a non-contact radio-frequency heating unit to the bladder of a patient.

In various embodiments, the control unit includes a resonant inverter, such as but not limited to a Class E resonant inverter. In various embodiments, the Class E resonant inverter further comprises a wide-bandgap transistor, and/or wherein the signal driving the gate of the transistor comprising the Class E resonant inverter is supplied by the microcontroller. In various embodiments, the supply to the Class E resonant inverter is the unfiltered rectified line voltage.

In various embodiments, the input voltage of the resonant electrical waveform generator is configured to vary in time at the fundamental of the line frequency (50 Hz or 60 Hz), and as a result the current drawn by the electrical waveform generator scales with voltage. If the voltage at the input of the electrical waveform generator is allowed to drop nearly to zero in sync with the rectified line, the electrical waveform generator itself may present approximately a resistive load to the line, and therefore nearly unity power factor can be achieved without any active or passive filtering elements.

In various embodiments, one or more of the temperature sensors configured to provide an output signal to the control unit may be read by the control circuitry during an OFF cycle of the modulation of the electrical output waveform(s) being provided to the electrode output terminal of the control unit. Temperature (and other) measurements may be susceptible to noise from switching power converters. Incorporation of temperature sensor(s) that can be read during the off-cycle modulation such that no switching noise is present, greatly improving the accuracy of the temperature reading. In various embodiments, one or more of the temperature sensors configured to provide an output signal to the control unit may be read during a minimum voltage level of the rectified line voltage. The gating of the resonant electrical waveform generator is disabled at the minimum rectified line voltage at the point of approximately zero power such that the temperature reading and off period due not adversely affect the power factor characteristics of the system.

In various embodiments, the ON and OFF switching cycles and the modulation periods may be synchronized with the line voltage or other electrical power input provided to the control unit. In various embodiments, the principal AC frequency; or the duty cycle of the transistor gate drive signal; are adjusted so as to optimize the heating efficiency; delivered power; or the power factor of the apparatus. As input voltage to the resonant electrical waveform generator varies with the rectified line voltage or otherwise, the optimal switching frequency and/or duty cycle may be affected, leading to reduced efficiency, and/or power factor. Varying frequency and/or duty cycle can lead to optimal efficiency and power factor for a given instantaneous input voltage or load impedance. Frequency and/or duty cycle may also be used to control power delivered to the load by deliberately tuning/detuning the impedance seen by the electrical waveform generator.

In various embodiments, the impedance characteristics of the liquid being heated is sensed by the control unit, and a resultant temperature is determined based on said impedance values. The impedance of saline changes very predictably with temperature. By monitoring the electrical performance of the electrical waveform generator during operation, the impedance can be determined and thus real-time temperature of the liquid can be predicted.

Various examples including and transporting the heated liquid into a human body where in the vicinity of the entry orifice of said liquid into the body, the temperature of the liquid is above that of the body.

In various embodiments the control unit in conjunction with a non-contact radio-frequency heating element may utilize high voltage DC pulses to transfer electrical energy into a liquid. The process of pulse electric field sterilization (PEF) is a method of applying high voltage DC pulses (could be bipolar DC voltages) to a liquid in order to destroy the cell walls of any bacteria that may be present within the liquid. In addition to sterilizing the liquid, the temperature of the liquid also increases moderately. PEF can be used to both sterilize and heat the liquid in real-time. The DC pulses can be on the order of 1 microsecond or greater in length. Generally, electric field strengths of 800 V/mm or higher are desired to achieve significant bacteria reduction in a liquid. The electrode strategy is similar to the AC method but in various embodiments may have exposed electrodes in contact with the liquid, such as metal electrodes.

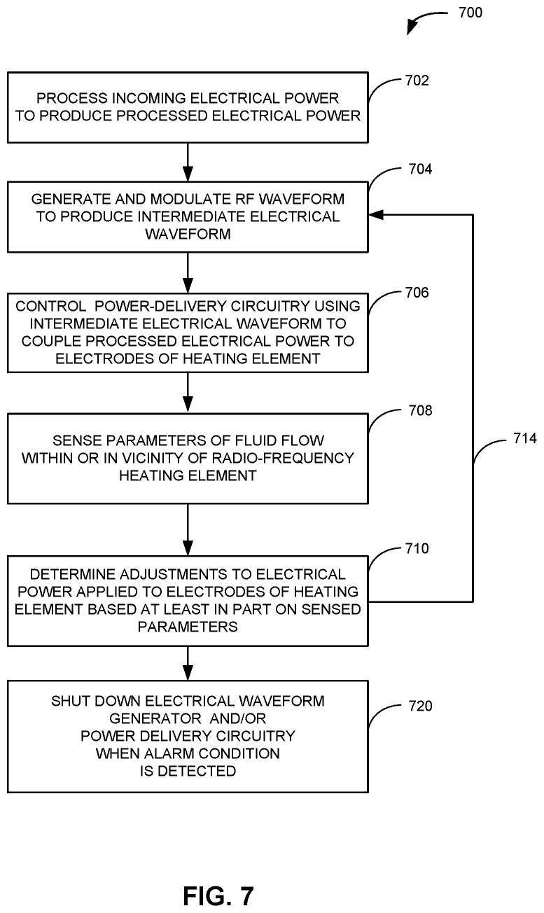

FIG. 7 illustrates a flowchart for a method 700 for non-contact radio-frequency heating control according to at least one embodiment. Embodiments of method 700 may utilize one or more, or any combination thereof, of devices and circuitry described above and throughout this disclosure, and any equivalents thereof, to perform the procedures and processes included as part of method 700. One, some, or all of the method steps described below, and any equivalents thereof, may be performed under the control of or based on control signals provided by control circuitry, such as but not limited to control circuitry 210 (FIGS. 2 and 4) including one or more processors, such as processor 211 (FIGS. 2 and 4).

Embodiments of method 700 may include processing incoming electrical power to produce processed electrical power, (block 702). Processing of electrical power may include rectification, filtering, and voltage, current, and/or phase regulation of incoming electrical power. In various embodiments, processor(s) of the control circuitry may provide control signals, for example to an input power processing circuitry, to modify or control one or more parameters, such as a voltage or a power level provided an output of the electrical power processed and provided as an output from the input power processing circuitry.

Embodiments of method 700 may include generating and modulating an RF waveform to produce an intermediate electrical waveform, (block 704). Generation of an RF waveform may be performed by an RF source, such as RF source 204A (FIGS. 2 and 4), and modulation of the RF waveform to produce the intermediate electrical waveform may be performed by a modulator, such as modulator 204B (FIGS. 2 and 4). The format and/or other parameters of the intermediate electrical waveform may correspond to any of the formats and may include any of the parameters described throughout this disclosure with respect to intermediate electrical waveforms, and any equivalents thereof.

Embodiments of method 700 may include controlling a power-delivery circuitry using the intermediate electrical waveform to control coupling of the processed electrical power to one or more sets of electrodes of a RF heating element (block 706). In various embodiments, the control provided by power-delivery circuitry may include switching ON and switching OFF the electrical coupling between the processed electrical power being provided to the power-delivery circuitry and the one or more sets of electrodes in order to provide a modulated or a pulsed electrical output waveform to the one or more sets of electrode included in the RF heating element, and thereby control the heating of a fluid flowing through or contained within the heating element. Embodiments of method 700 may include coupling the electrical output waveform from the one or more electrode output terminals of a control unit to one or more sets of electrodes positioned in a non-contact radio-frequency heating elements through one or more electrical conductors, such as but not limited to one or more shielded co-axial cables. Providing the electrical output waveform from the one or more electrode output terminals to one or more sets of electrodes positioned in a non-contact radio-frequency heating element may result in a heating of a fluid passing between or contained within an area between the one or more sets of electrodes.

Embodiments of method 700 may include sensing parameters associated with a fluid flowing through and/or contained within the RF heating element (block 708). Sensed parameter may include but are not limited to sensing a temperature of the fluid, sensing a flow rate or a flow volume associated with the fluid, and/or sensing of a impedance value of the electrical circuit including the electrodes and the fluid present within the area between the electrodes.

Embodiments of method 700 may include determining, for example using control circuitry, adjustments to the electrical power that is/are being applied to the one or more sets of electrodes of the non-contact radio-frequency heating element based at least in part on the sensed parameters (block 710). In various embodiments, a determination of any adjustments to the electrical output waveform(s) is based on one or more sensed temperatures associated with the fluid being heated by the non-contact radio-frequency heating element. In various embodiments, a determination of any adjustments to the electrical output waveform(s) is based on one or more sensed flow rates or a sensed flow volume associated with the fluid being heated by the non-contact radio-frequency heating element. In various embodiments, a determination of any adjustments to the electrical output waveform(s) is based on one or more sensed impedance values measured for the electrical circuitry that includes the electrodes of the non-contact radio-frequency heating element and the fluid being heated by the non-contact radio-frequency heating element.

Based on a determination of any adjustment that may need to be made to the electrical output waveform(s), method 700 returns to block 704, as indicated by return line 714, where further control of the electrical waveform generator and/or the power-delivery circuitry is performed based on any of the adjustments determined to be made at block 710. Heating of the fluid may continue in a loop including blocks 702 through 710 until an operator shuts the control unit that is performing method 700 off, or an alarm condition is detected (block 720).

When an alarm condition is detected, embodiments of method 700 may include shutting down the electrical waveform generator and/or the power-delivery circuitry of the control unit. Alarm conditions can include but are not limited to an over temperature detected for the fluid being heated by the non-contact radio-frequency heating element, an unacceptable condition related to the flow rate or flow volume associated with the fluid being heated by the non-contact radio-frequency heating unit, and/or an electrical or temperature condition associated with the control unit and/or the non-contact radio-frequency heating unit, such as a short circuit, loss of input electrical power to the control unit, and any unacceptable and detected over temperature, overcurrent, or overvoltage condition that might exist within the control unit. In various embodiments, the control unit is configured to output a warning signal, for example through a user interface, to one or more devices located external to the control unit, the warning signal(s) including information related to the detection of an alarm condition, and/or information related to the nature and the extent of the condition that generated the alarm condition.

As will be appreciated, aspects of the disclosure may be embodied as a system, method or program code/instructions stored in one or more machine-readable media. Accordingly, aspects may take the form of hardware, software (including firmware, resident software, micro-code, etc.), or a combination of software and hardware aspects that may all generally be referred to herein as a “circuit,” “circuitry,” “module,” or “system.” The functionality presented as individual modules/units in the example illustrations can be organized differently in accordance with any one of platform (operating system and/or hardware), application ecosystem, interfaces, programmer preferences, programming language, administrator preferences, etc.

Any combination of one or more machine readable medium(s) may be utilized. The machine readable medium may be a machine readable signal medium or a machine readable storage medium. A machine readable storage medium may be, for example, but not limited to, a system, apparatus, or device, that employs any one of or combination of electronic, magnetic, optical, electromagnetic, infrared, or semiconductor technology to store program code. More specific examples (a non-exhaustive list) of the machine readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a machine readable storage medium may be any tangible medium that can contain, or store a program for use by or in connection with an instruction execution system, apparatus, or device. A machine readable storage medium is not a machine readable signal medium.

While the aspects of the disclosure are described with reference to various implementations and exploitations, it will be understood that these aspects are illustrative and that the scope of the claims is not limited to them. In general, techniques for non-contact radio-frequency heating control units and non-contact radio-frequency heating elements as described herein may be implemented with facilities consistent with any hardware system or hardware systems. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” can be infringed with only one of the listed items, multiple of the listed items, and one or more of the items in the list and another item not listed.

Example embodiments include the following.

Embodiment 1. An apparatus comprising: a control unit for heating a fluid within a non-contact radiofrequency heating element, the control unit comprising: a radio-frequency (RF) source configured to generate an electrical waveform; a modulator coupled to the RF source and configured to receive the electrical waveform from the RF source and to generate an intermediate electrical waveform having a waveform based at least in part on the electrical waveform; and a power-delivery circuitry coupled to the modulator and configured to receive an electrical power input and to receive the intermediate electrical waveform, and a to generate an electrical output waveform using the electrical power input, the electrical output waveform corresponding to the intermediate electrical waveform; wherein the power-delivery circuitry is configured to deliver the electrical output waveform to a set of electrodes included as part of the non-contact radiofrequency heating element, the set of electrodes positioned proximate to a fluid flow passageway of the radiofrequency heating element while not in fluid communication with the fluid flow passageway, and wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

Embodiment 2. The apparatus of embodiment 1, wherein the control unit further comprises a control circuitry including at least one processor, wherein the at least one processor is communicatively coupled to the modulator, and wherein the at least one processor is configured to provide one or more control signals to the modulator to control at least one parameter of the intermediate electrical waveform generated by the modulator.

Embodiment 3. The apparatus of embodiment 2, wherein controlling at least one parameter of the intermediate electrical waveform includes controlling a duty cycle of the intermediate electrical waveform.

Embodiment 4. The apparatus of embodiment 2, wherein the control unit further comprises one or more sensor inputs, the one or more sensor inputs coupled to the control circuitry and configured to receive one or more sensor signals generated by one or more sensors, and wherein the at least one processor is configured to process the one or more sensor signals, and to generate the one or more control signals provided to the modulator based at least in part on the one or more sensor signals.

Embodiment 5. The apparatus of embodiment 4, wherein at least one of the one or more sensor signals is generated by a temperature sensor, and is configured to provide a signal corresponding to a sensed temperature of the fluid flowing through or exiting the non-contact radiofrequency heating element.

Embodiment 6. The apparatus of embodiment 4, wherein at least one of the one or more sensor signals is generated by a flow sensor, and is configured to provide a signal corresponding to a sensed flow rate of the fluid flowing through or exiting the non-contact radiofrequency heating element.

Embodiment 7. The apparatus of embodiment 4, wherein at least one of the one or more sensor signals is generated by a ambient temperature sensor, and is configured to provide a signal corresponding to a sensed temperature of an ambient area where a fluid source configured to provide the fluid flow to the non-contact radiofrequency heating element is located.

Embodiment 8. The apparatus of any of embodiments 1 to 7, wherein the electrical output waveform generated by the electrical waveform generator has a frequency in a range of 10 kilohertz to 30 megahertz, inclusive.

Embodiment 9. The apparatus of any of the embodiments 1 to 8, wherein at least some portion of the electrical output waveform configured to be delivered to the set of electrodes comprises a sine wave.

Embodiment 10. The apparatus of any of embodiments 1 to 9, wherein the electrical output waveform is configured to produce volumetric radiofrequency heating in a fluid comprising saline.

Embodiment 11. The apparatus of any of embodiments 1 to 10, wherein the power-delivery circuitry comprises one or more electrical switching devices configured to controllably connect and disconnect the electrical output waveform to and from, respectively, the set of electrodes.

Embodiment 12. The apparatus of any of embodiments 1 to 11, wherein the control unit comprises a plurality of electrode output terminals, each of the plurality of electrode output terminals configured to be electrically coupled to a separate one of the set of electrodes included as part of the non-contact radiofrequency heating element, and wherein the power-delivery circuitry is configured to connect and disconnect the electrical output waveform to and from, respectively, each of the plurality of electrode output terminals individually.

Embodiment 13. A system comprising: a non-contact radiofrequency heating element comprising a set of electrodes arranged proximate to a fluid passageway extending through the non-contact radiofrequency heating element, the set of electrodes physically isolated from the fluid passageway by a barrier and configured to produce non-contact radio-frequency heating in a fluid flowing through the fluid passageway when an electrical output waveform is applied to the set of electrodes; and a control unit coupled to the non-contact radiofrequency heating element and configured to provide the electrical output waveform for heating the fluid flowing within the non-contact radiofrequency heating element, the control unit comprising: a radio-frequency (RF) source configured to generate an electrical waveform; a modulator coupled to the RF source and configured to receive the electrical waveform from the RF source and to generate an intermediate electrical waveform having a waveform based at least in part on the electrical waveform; and a power-delivery circuitry coupled to the modulator and configured to receive an electrical power input and to receive the intermediate electrical waveform, and a to generate an electrical output waveform from using the electrical power input, the electrical output waveform corresponding to the waveform of the intermediate electrical waveform; wherein the power-delivery circuitry is configured to deliver the electrical output waveform to the set of electrodes included as part of the non-contact radiofrequency heating element, and wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

Embodiment 14. The system of embodiment 13, wherein the non-contact radiofrequency heating control unit further comprises one or more sensor inputs, the one or more sensor inputs coupled to the control unit and configured to receive one or more sensor signals generated by one or more sensors, and to couple the one or more sensor signals to at least one processor, wherein the processor is configured to process the one or more sensor signals, and to generate one or more control signals provided to the modulator to control the generation of the intermediate electrical waveform based at least in part on the sensor signals.

Embodiment 15. The system of embodiment 14, wherein at least one of the one or more sensor signals is generated by a temperature sensor, and is configured to provide a signal corresponding to a sensed temperature of the fluid flowing through or exiting the non-contact radiofrequency heating element.

Embodiment 16. The system of any of embodiments 13 to 15, wherein at least some portion of the intermediate electrical waveform generated by the modulator comprises a frequency in a range of 10 kilohertz to 30 megahertz, inclusive.

Embodiment 17. The system of any of embodiments 13 to 16, wherein the intermediate electrical waveform generated by the modulator comprises a sine wave.

Embodiment 18. The system of any of embodiments 13 to 17, wherein the control unit is configured to control the non-contact radio-frequency heating of a flow of a saline fluid flowing through the non-contact radiofrequency heating element.

Embodiment 19. The system of any of embodiments 13 to 18, wherein the power-delivery circuitry comprises one or more electrical switching devices configured to controllably connect and disconnect the electrical output waveform to and from, respectively, the set of electrodes.

Embodiment 20. The system of any of embodiments 13 to 19, wherein the control unit comprises a plurality of electrode output terminals, each of the plurality of electrode output terminals configured to be electrically coupled to a separate one of the set of electrodes included as part of the non-contact radiofrequency heating element, and wherein the power-delivery circuitry is configured to connect and disconnect the electrical output waveform to and from, respectively, each of the plurality of electrode output terminals individually.

Embodiment 21. A method comprising: generating a radiofrequency(RF) waveform; modulating the RF waveform to produce and intermediate electrical waveform; and controlling a power-delivery circuitry using the intermediate electrical waveform to controllably couple an electrical output waveform to a set of electrodes included as part of a radiofrequency heating element, the set of electrodes positioned proximate to a fluid flow passageway of the radiofrequency heating element while not in fluid communication with the fluid flow passageway; wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

Embodiment 22. The method of embodiment 21, further comprising: receiving a sensor output signal corresponding to a sensed temperature of the fluid; and adjusting at least one parameter of the electrical output waveform being applied to the set of electrodes based at least in part on the sensed temperature.

Embodiment 23. The method of embodiment 22, wherein adjusting the at least one parameter of the electrical output waveform comprises adjusting a duty cycle of electrical output waveform.

Embodiment 24. The method of embodiment 22, wherein adjusting the at least one parameter of the electrical output waveform comprises adjusting a voltage level of the electrical output waveform.

Embodiment 25. The method of embodiment 22, wherein adjusting the at least one parameter of the electrical output waveform comprises adjusting a frequency of the electrical output waveform.

Embodiment 26. The method of any of embodiments 21 to 25, wherein at least some portion of the electrical output waveform comprises a sine wave having a frequency in a range of 10 kilohertz to 30 megahertz, inclusive.

Embodiment 27. The method of any of embodiments 21 to 26, wherein the set of electrodes comprises four electrodes and at least one return electrode.

Embodiment 28. The method of any of embodiments 21 to 27, further comprising: monitoring a sensor signal corresponding to a temperature of the fluid being heated, determining that the temperature of the fluid has exceeded a temperature threshold value based on the sensor signal; and controlling a shutdown of the modulator or a power-delivery circuitry so that the electrical output waveform is no longer applied to the set of electrodes.

Embodiment 29. A non-transitory, computer-readable medium having instructions stored thereon that are executable by a computing device to perform operations comprising: generating a radiofrequency(RF) waveform; modulating the RF waveform to produce and intermediate electrical waveform; and controlling a power-delivery circuitry using the intermediate electrical waveform to controllably couple an electrical output waveform to a set of electrodes included as part of a radiofrequency heating element, the set of electrodes positioned proximate to a fluid flow passageway of the radiofrequency heating element while not in fluid communication with the fluid flow passageway; wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

Embodiment 30. The non-transitory, computer-readable medium of embodiment 29, further comprising: monitoring a sensor signal corresponding to a temperature of the fluid being heated, determining that the temperature of the fluid has exceeded a temperature threshold value based on the sensor signal; and shutting down the power-delivery circuitry so that the electrical output waveform is no longer applied to the set of electrodes.

1. An apparatus comprising:
a control unit for heating a fluid within a non-contact radiofrequency heating element, the control unit comprising:
a radio-frequency (RF) source configured to generate an electrical waveform;
a modulator coupled to the RF source and configured to receive the electrical waveform from the RF source and to generate an intermediate electrical waveform having a waveform based at least in part on the electrical waveform; and
a plurality of electrode output terminals, each of the plurality of electrode output terminals configured to be electrically coupled to a separate one of a set of electrodes included as part of the non-contact radiofrequency heating element;
a power-delivery circuitry coupled to the modulator and configured to receive an electrical power input and to receive the intermediate electrical waveform, and a to generate an electrical output waveform using the electrical power input, the electrical output waveform corresponding to the intermediate electrical waveform;
wherein the power-delivery circuitry is configured to deliver the electrical output waveform to the set of electrodes included as part of the non-contact radiofrequency heating element, the set of electrodes positioned proximate to a fluid flow passageway of the radiofrequency heating element while not in fluid communication with the fluid flow passageway,
wherein the electrical output waveform is configured to produce volumetric radiofrequency heating in a fluid contained within the fluid flow passageway when the electrical output waveform is being applied to a set of electrodes and while the set of electrodes is not in physical contact with the fluid, and
wherein the power-delivery circuitry is configured to connect and disconnect the electrical output waveform to and from, respectively, each of the plurality of electrode output terminals individually.

2. The apparatus of claim 1, wherein the control unit further comprises a control circuitry including at least one processor,
wherein the at least one processor is communicatively coupled to the modulator, and
wherein the at least one processor is configured to provide one or more control signals to the modulator to control at least one parameter of the intermediate electrical waveform generated by the modulator.

3. The apparatus of claim 2, wherein controlling at least one parameter of the intermediate electrical waveform includes controlling a duty cycle of the intermediate electrical waveform.

4. The apparatus of claim 2, wherein the control unit further comprises one or more sensor inputs, the one or more sensor inputs coupled to the control circuitry and configured to receive one or more sensor signals generated by one or more sensors, and
wherein the at least one processor is configured to process the one or more sensor signals, and to generate the one or more control signals provided to the modulator based at least in part on the one or more sensor signals.

5. The apparatus of claim 4, wherein at least one of the one or more sensor signals is generated by a temperature sensor, and is configured to provide a signal corresponding to a sensed temperature of the fluid flowing through or exiting the non-contact radiofrequency heating element.

6. The apparatus of claim 4, wherein at least one of the one or more sensor signals is generated by a flow sensor, and is configured to provide a signal corresponding to a sensed flow rate of the fluid flowing through or exiting the non-contact radiofrequency heating element.

7. The apparatus of claim 4, wherein at least one of the one or more sensor signals is generated by a ambient temperature sensor, and is configured to provide a signal corresponding to a sensed temperature of an ambient area where a fluid source configured to provide the fluid flow to the non-contact radiofrequency heating element is located.

8. The apparatus of claim 1, wherein the electrical output waveform generated by the electrical waveform generator has a frequency in a range of 10 kilohertz to 30 megahertz, inclusive.

9. The apparatus of claim 1, wherein at least some portion of the electrical output waveform configured to be delivered to the set of electrodes comprises a sine wave.

10. The apparatus of claim 1, wherein the electrical output waveform is configured to produce volumetric radiofrequency heating in a fluid comprising saline.

11. The apparatus of claim 1, wherein the power-delivery circuitry comprises one or more electrical switching devices configured to controllably connect and disconnect the electrical output waveform to and from, respectively, the set of electrodes.

12. (canceled)

13. A system comprising:
a non-contact radiofrequency heating element comprising a set of electrodes arranged proximate to a fluid passageway extending through the non-contact radiofrequency heating element, the set of electrodes physically isolated from the fluid passageway by a barrier and configured to produce non-contact radio-frequency heating in a fluid flowing through the fluid passageway when an electrical output waveform is applied to the set of electrodes; and
a control unit coupled to the non-contact radiofrequency heating element and configured to provide the electrical output waveform for heating the fluid flowing within the non-contact radiofrequency heating element, the control unit comprising:
a radio-frequency (RF) source configured to generate an electrical waveform;
a modulator coupled to the RF source and configured to receive the electrical waveform from the RF source and to generate an intermediate electrical waveform having a waveform based at least in part on the electrical waveform; and
a plurality of electrode output terminals, each of the plurality of electrode output terminals configured to be electrically coupled to a separate one of the set of electrodes included as part of the non-contact radiofrequency heating element;
a power-delivery circuitry coupled to the modulator and configured to receive an electrical power input and to receive the intermediate electrical waveform, and a to generate an electrical output waveform from using the electrical power input, the electrical output waveform corresponding to the waveform of the intermediate electrical waveform;
wherein the power-delivery circuitry is configured to deliver the electrical output waveform to the set of electrodes included as part of the non-contact radiofrequency heating element,
wherein the electrical output waveform is configured to produce volumetric radiofrequency heating in a fluid contained within the fluid flow passageway when the electrical output waveform is being applied to a set of electrodes and while the set of electrodes is not in physical contact with the fluid, and
wherein the power-delivery circuitry is configured to connect and disconnect the electrical output waveform to and from, respectively, each of the plurality of electrode output terminals individually.

14. The system of claim 13, wherein the non-contact radiofrequency heating control unit further comprises one or more sensor inputs, the one or more sensor inputs coupled to the control unit and configured to receive one or more sensor signals generated by one or more sensors, and to couple the one or more sensor signals to at least one processor,
wherein the processor is configured to process the one or more sensor signals, and to generate one or more control signals provided to the modulator to control the generation of the intermediate electrical waveform based at least in part on the sensor signals.

15. The system of claim 14, wherein at least one of the one or more sensor signals is generated by a temperature sensor, and is configured to provide a signal corresponding to a sensed temperature of the fluid flowing through or exiting the non-contact radiofrequency heating element.

16. The system of claim 13, wherein at least some portion of the intermediate electrical waveform generated by the modulator comprises a frequency in a range of 10 kilohertz to 30 megahertz, inclusive.

17. The system of claim 13, wherein the intermediate electrical waveform generated by the modulator comprises a sine wave.

18. The system of claim 13, wherein the control unit is configured to control the non-contact radio-frequency heating of a flow of a saline fluid flowing through the non-contact radiofrequency heating element.

19. The system of claim 13, wherein the power-delivery circuitry comprises one or more electrical switching devices configured to controllably connect and disconnect the electrical output waveform to and from, respectively, the set of electrodes.

20. (canceled)

21. A method comprising:
generating a radiofrequency(RF) waveform;
modulating the RF waveform to produce and intermediate electrical waveform; and
controlling a power-delivery circuitry using the intermediate electrical waveform to controllably connect and disconnect an electrical output waveform to and from, respectively, a plurality of electrode output terminals each electrically coupled to a separate one of a set of electrodes included as part of a radio frequency heating element, the set of electrodes positioned proximate to a fluid flow passageway of the radiofrequency heating element while not in fluid communication with the fluid flow passageway;
wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

22. The method of claim 21, further comprising:
receiving a sensor output signal corresponding to a sensed temperature of the fluid; and
adjusting at least one parameter of the electrical output waveform being applied to the set of electrodes based at least in part on the sensed temperature.

23. The method of claim 22, wherein adjusting the at least one parameter of the electrical output waveform comprises adjusting a duty cycle of electrical output waveform.

24. The method of claim 22, wherein adjusting the at least one parameter of the electrical output waveform comprises adjusting a voltage level of the electrical output waveform.

25. The method of claim 22, wherein adjusting the at least one parameter of the electrical output waveform comprises adjusting a frequency of the electrical output waveform.

26. The method of claim 21, wherein at least some portion of the electrical output waveform comprises a sine wave having a frequency in a range of 10 kilohertz to 30 megahertz, inclusive.

27. The method of claim 21, wherein the set of electrodes comprises four electrodes and at least one return electrode.

28. The method of claim 21, further comprising:
monitoring a sensor signal corresponding to a temperature of the fluid being heated,
determining that the temperature of the fluid has exceeded a temperature threshold value based on the sensor signal; and
controlling a shutdown of the modulator or a power-delivery circuitry so that the electrical output waveform is no longer applied to the set of electrodes.

29. A non-transitory, computer-readable medium having instructions stored thereon that are executable by a computing device to perform operations comprising:
generating a radiofrequency(RF) waveform;
modulating the RF waveform to produce and intermediate electrical waveform; and
controlling a power-delivery circuitry using the intermediate electrical waveform to controllably connect and disconnect an electrical output waveform to and from, respectively, a plurality of electrode output terminals each electrically coupled to a separate one of a set of electrodes included as part of a radiofrequency heating element, the set of electrodes positioned proximate to a fluid flow passageway of the radiofrequency heating element while not in fluid communication with the fluid flow passageway;
wherein the electrical output waveform is configured to produce volumetric radiofrequency heating in a fluid contained within the fluid flow passageway when the electrical output waveform is being applied to a set of electrodes and while the set of electrodes is not in physical contact with the fluid.

30. The non-transitory, computer-readable medium of claim 29, further comprising:
monitoring a sensor signal corresponding to a temperature of the fluid being heated,
determining that the temperature of the fluid has exceeded a temperature threshold value based on the sensor signal; and
shutting down the power-delivery circuitry so that the electrical output waveform is no longer applied to the set of electrodes.
